(19) United States
(12) Patent Application Publication
(10) Pub. No.: US 2008/0221203 A1
(43) Pub. Date: Sep. 11, 2008

(54) Use of Agonists and Antagonists of Beta Adrenoceptors for Treating Arterial Disease

(75) Inventors: Chantal Dessy, Brussel (BE); Jean-Luc Balligand, Brussel (BE)

Correspondence Address: KNOBBE MARTENS OLSON & BEAR LLP, 2040 MAIN STREET, FOURTEENTH FLOOR, IRVINE, CA, 92614, US

(73) Assignee: UNIVERSITY CATHOLIQUE DE LOUVAIN, Louvain-la-Neuve (BE)

(21) Appl. No.: 11/659,896

(22) PCT Filed: Aug. 8, 2005
(86) PCT No.: PCT/EP05/08569

§ 371 Date: May 23, 2008

Related U.S. Application Data
(60) Provisional application No. 60/600,093, filed on Aug. 9, 2004.

(51) Int. Cl. A61K 31/352; A61P 9/04

(52) U.S. Cl. 514/456

(57) ABSTRACT

Methods for treating and/or preventing cardiovascular diseases and diseases related thereto by administering a therapeutically effective amount of one or more first compound(s) having a beta3-adrenoceptor agonistic effect and a therapeutically effective amount of one or more second compound(s) having a beta1/beta2-adrenoceptor antagonistic effect are disclosed. The first compound(s) and the second compound(s) are used simultaneously, separately or sequentially as a combined preparation. Related cardiovascular diseases include arterial diseases, ischemic and failing cardiac diseases, including heart failure, conditions related to metabolic syndrome or angiogenesis-related disease. Pharmaceutical compositions are also described.

### FIELD OF THE INVENTION

The present invention relates to the medical field. The present invention relates to the use of agonists and antagonists of beta-adrenoceptors for treating cardiovascular diseases including arterial diseases such as coronary, peripheral and cerebral artery diseases and for treating ischemic and failing cardiac diseases and/or diseases related thereto. The present invention also relates to the use of agonists and antagonists of beta-adrenoceptors for treating conditions related to, or that may cause cardiovascular diseases including conditions related to metabolic syndrome. The present invention also relates to a methods and compositions for treating said diseases.

### BACKGROUND OF THE INVENTION

Cardiovascular diseases (CVD) are diseases which affect the heart (cardio) and/or the body's entire system of blood vessels (vascular). A large number of conditions are classified as types of CVD. Many of these are linked to a build-up of fatty plaques in blood vessels (for example, coronary heart disease, most strokes, peripheral vascular disease). The list of diseases that are considered CVD includes, but is not limited to, hypertension, atherosclerosis, cerebrovascular diseases, peripheral vascular disease, and heart diseases such as coronary heart disease (CHD) and coronary artery disease (CAD), the most common forms of heart disease, eventually leading to angina (chest pain) or heart attack (myocardial infarction), heart failure, including congestive heart failure (CHF), cardiomyopathy (abnormality of the heart muscle), pericardial disease, etc. The vascular system is made up of the vessels that carry the blood. Arteries carry oxygen-rich blood away from the heart. Veins carry oxygen-poor blood back to the heart. Diseases of circulatory systems, herein also referred to as arterial diseases, are diseases wherein optimal functioning of the arteries is affected, resulting in a sub-optimal and insufficient blood flow. Different types of arterial diseases can be distinguished.

Coronary artery disease (CAD) and peripheral artery disease (PAD) are conditions characterized by insufficient blood flow, usually secondary to atherosclerosis. Symptoms of ischemia (angina pectoris for CAD or intermittent claudication for PAD) are brought on by stress and relieved by rest. In CAD, symptoms may become life threatening due to myocardial infarction, arrhythmia, and progressive heart failure. In PAD, symptoms are less likely to be life threatening except when critical limb ischemia develops, but the risk of adverse cardiovascular events and death is increased.

Identification and management of risk factors are important in the medical management of both CAD and PAD. Pharmacologic management of risk factors may include anti-hypertensives, lipid-lowering agents, and hypoglycemic agents; smoking cessation, diet, and exercise are often prescribed with variable compliance. Pharmacologic management aimed at reduction of symptoms of ischemia often includes vasodilators, anti-anginal, and anti-platelet therapy. Mechanical revascularization by percutaneous angioplasty (with or without a stent) and direct surgical reconstruction improve blood flow and reduce symptoms. However, restenosis after angioplasty and progression of disease may limit the duration of the benefit.

PAD afflicts approximately 11 million patients in the United States. Approximately one third of these patients experience intermittent claudication (discomfort, pain, fatigue, or heaviness in the leg muscles that consistently is brought on by the same amount of muscular activity and relieved by rest). Claudication is similar to angina and represents ischemic muscle pain that may be localized to the hip, buttock, thigh, or calf. It occurs predictably with the same amount of physical stress. Atherosclerosis is systemic, but often one lower limb is more affected than the other. Patients may develop critical limb ischemia, with rest pain, non-healing ulcers, and/or gangrene. Rest pain occurs when blood supply is inadequate to meet the basic nutritional requirements at rest and typically localizes in the toes or foot of the affected limb.

The prevalence of CAD and PAD is expected to increase in countries with aging populations, as aging is a primary risk factor for atherosclerosis. Less invasive catheter-based treatment methods and more cost-effective programs and treatment methodologies are needed to manage these conditions.

Heart failure is a condition in which the heart has lost the ability to pump enough blood to the body's tissues. With too little blood being delivered, the organs and other tissues do not receive enough oxygen and nutrients to function properly. Often, a person with heart failure may have a buildup of fluid in the tissues, called edema. Heart failure with this kind of fluid buildup is generally called congestive heart failure.

Amongst the causes for cardiovascular diseases it is worth mentioning the metabolic syndrome. Patients who have this syndrome have been shown to be at an increased risk of developing cardiovascular disease. Metabolic syndrome is a common condition that goes by many names: dysmetabolic syndrome, syndrome X, insulin resistance syndrome, obesity syndrome, and Reaven's syndrome. Metabolic syndrome is a set of risk factors that includes: abdominal obesity, a decreased ability to process glucose (insulin resistance), dyslipidemia (unhealthy lipid levels), low HDL and high LDL cholesterol levels, high triglyceride levels, abnormalities of blood clotting and hypertension.

There remains a need in the art for providing improved methods and compositions for treating these cardiovascular diseases and conditions leading to such diseases, such as e.g. syndrome X.

The present invention aims to provide improved methods and compositions for treating diseases of circulatory systems and of the heart.

### SUMMARY OF THE INVENTION

The present invention relates to the use of agonists and antagonists of beta-adrenoceptors for treating cardiovascular diseases and diseases related thereto. The present invention also relates to methods and compositions for treating said diseases.

The present invention is in part based on the Applicants' finding that compounds having a beta3-adrenoceptor agonistic effect improve coronary circulation. Compounds having a beta3-adrenoceptor agonistic effect can also mediate relaxation (vasodilatation) of peripheral arteries, e.g. the aorta, and cerebral arteries. More in particular, it was demonstrated that compounds having a beta3-adrenoceptor agonistic effect mediate relaxation (vasodilatation) of coronary arteries. As a result thereof, administration of this type of compounds permits to greatly improve perfusion of the heart muscle. Improved perfusion of the heart muscle beneficiates its integrity and functionally.

Moreover, when these compounds are administered in combination with one or more compound(s) having a β1, β2-adrenoceptor antagonistic effect, such combination provides a double effect: an improvement of perfusion of the heart muscle (vasodilating mediated by the beta3-adrenoceptor agonistic activity) and a reduction of the contraction force of the cardiac muscle (mediated by the beta1, beta2-adrenoceptor antagonistic activity). In view of such advantageous effect, the present compounds may be used in the treatment of various types of cardiovascular diseases, as indicated below.

In a first aspect, the present invention therefore relates to the use of one or more first compound(s) having a beta3-adrenoceptor agonistic effect and one or more second compound(s) having a beta1/beta2-adrenoceptor antagonistic effect for the preparation of a medicament for treating and/or preventing cardiovascular diseases and diseases related thereto, wherein said one or more first compound(s) and said one or more second compound(s) are used as combined preparation for simultaneous, separate or sequential use. More in particular, in one embodiment, said treatment and/or prevention is further defined as treating and/or preventing arterial diseases and/or diseases related thereto, and preferably said arterial diseases comprise coronary, peripheral or cerebral artery diseases. In another embodiment, said treatment and/or prevention is further defined as treating and/or preventing ischemic and failing cardiac diseases and/or diseases related thereto. Preferably, said failing cardiac disease comprises heart failure, and even more preferably diastolic heart failure. In yet another embodiment, said treatment and/or prevention is further defined as treating and/or preventing one or more conditions related to metabolic syndrome.

In a second aspect, the present invention relates to the use of a compound having a beta3-adrenoceptor agonistic effect for the preparation of a medicament for treating and/or preventing cardiovascular diseases and diseases related thereto. More in particular, in one embodiment, said treatment and/or prevention is further defined as treating and/or preventing arterial diseases and/or diseases related thereto, and preferably said arterial diseases comprise coronary, peripheral or cerebral artery diseases. In another embodiment, said treatment and/or prevention is further defined as treating and/or preventing ischemic and failing cardiac diseases and/or diseases related thereto. Preferably, said failing cardiac disease comprises heart failure, and even more preferably diastolic heart failure. In yet another embodiment, said treatment and/or prevention is further defined as treating and/or preventing one or more conditions related to metabolic syndrome.

In a third aspect, the invention relates to the use of (a) compound(s) according to the invention, for the preparation of a medicament wherein said compound(s) further stimulate(s) neo-angiogenesis. The invention further thus relates to the use of (a) compound(s) according to the invention, for the preparation of a medicament for stimulating neo-angiogenesis and/or for treating angiogenesis-related diseases. In one embodiment, the invention provides the use of a compound having a beta3-adrenoceptor agonistic effect. In another embodiment, the invention provides the use of a compound having a beta3-adrenoceptor agonistic effect and a beta1/beta2-adrenoceptor antagonistic effect. In yet another embodiment, the invention provides the use of a combination of one or more first compound(s) having a beta3-adrenoceptor agonistic effect with one or more second compound(s) having a beta1/beta2-adrenoceptor antagonistic as a combined preparation for simultaneous, separate or sequential use.

The invention further relates to compositions and methods which are effective, specific and which have limited side-effects for treating and/or preventing cardiovascular diseases and diseases related thereto, as defined above, and including in particular arterial diseases, and preferably coronary, peripheral and cerebral artery diseases, ischemic and failing cardiac diseases, angiogenesis-related diseases and/or diseases related thereto, and/or conditions related to metabolic syndrome.

Those skilled in the art will immediate recognize the many other effects and advantages of the present method and composition and the numerous possibilities for end uses of the present invention from the detailed description and examples provided below.

### DETAILED DESCRIPTION OF THE FIGURES

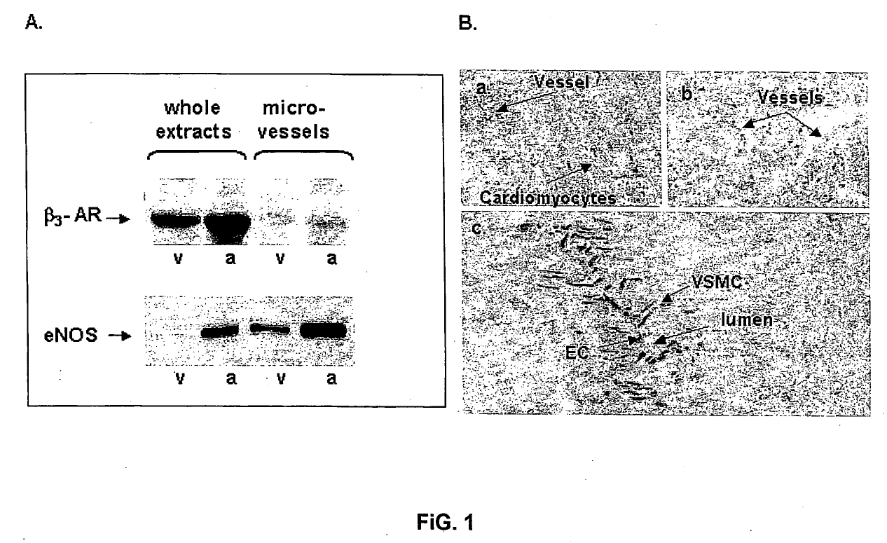

FIG. 1 illustrates endothelium-restricted expression of beta3-adrenoceptors in coronary microarteries. A. immunoblots for beta3-adrenoceptors (upper lane) and eNOS (lower lane) from protein homogenates of human coronary microarteries isolated from left ventricle (v) or right atria (a) and from protein homogenates prepared from whole left ventricular (v) and right atrial (a) pieces. Note that immunodetected signals are consistently stronger in extracts from atria. This blot is representative of at least 3 similar experiments. B. immunostaining for beta3-adrenoceptors in human right atrial appendages; a. lower magnification; b. negative control obtained in the absence of specific antibodies; c. longitudinal section of microartery at higher magnification. These results are representatives of 6 similar experiments.

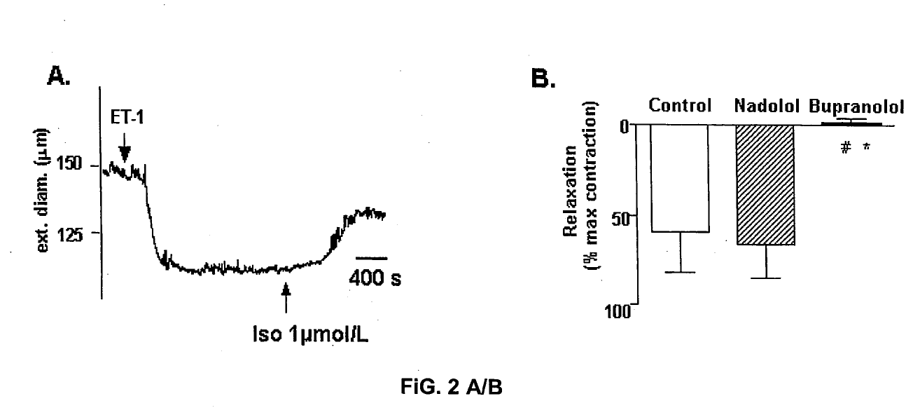

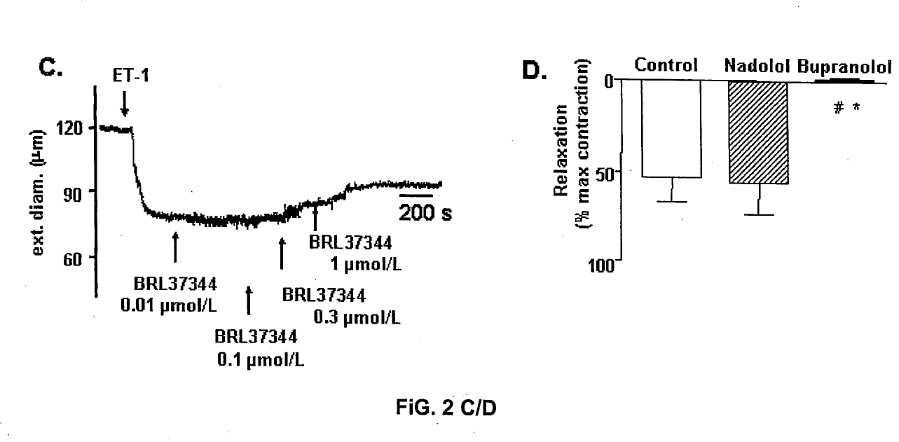

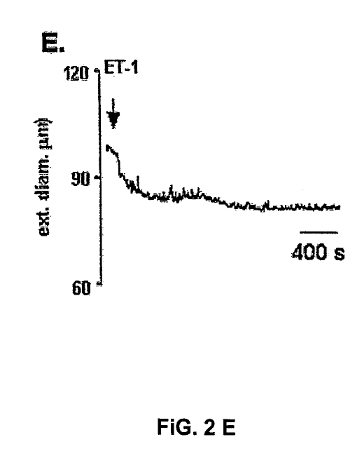

FIG. 2 shows that beta-agonist mediated relaxation of coronary microarteries involves a beta3-adrenoceptor pathway. A. Representative tracing showing the isoprenaline-evoked relaxation of a human coronary microarteriole constricted with ET-1; B. Isoprenaline evoked-relaxations of isolated human coronary microarteries constricted with ET-1 in the absence (open column) or the presence of nadolol (hatched column) or bupranolol (mean results ±sem are expressed as % of the maximum ET-1-evoked constriction, n=3-5). C. Representative tracing showing the dose-dependent relaxation to the beta3-preferential agonist BRL37344 of a human coronary microarteriole constricted with ET-1. D. BRL37344 evoked-relaxations of isolated human coronary microarteries constricted with ET-1 in the absence (open column) or the presence of nadolol (hatched column) or bupranolol (mean results sem are expressed as % of the maximum ET-1-evoked contraction, n=4-6). *, P<0.05 vs. control; #, P<0.05 vs. nadolol by ANOVA. The relaxation to both agonists is resistant to beta₁₋₂-blockade with nadolol, but abolished with beta₁₋₂₋₃-blockade with bupranolol, demonstrating a beta₃-mediated relaxation. E. Typical tracing depicting the sustained contraction with ET-1 alone of a human coronary microartery over similar time intervals (time control).

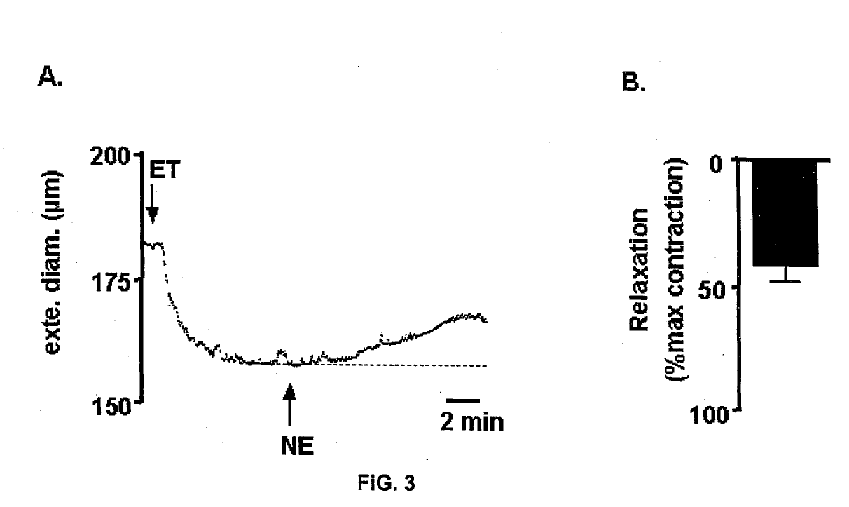

FIG. 3 illustrates that norepinephrine evokes a beta₃-mediated relaxation of coronary microarteries. A. Representative tracing showing the relaxation to norepinephrine (1 μmol/l) of a human coronary microarteriole constricted with ET-1 in the presence of an alpha₁₋₂ blocker (phentolamine) and a beta₁₋₂ blocker (nadolol). B. Quantification of the norepinephrine relaxation; results ±sem are expressed as % of the maximum ET-1 contraction, n=3).

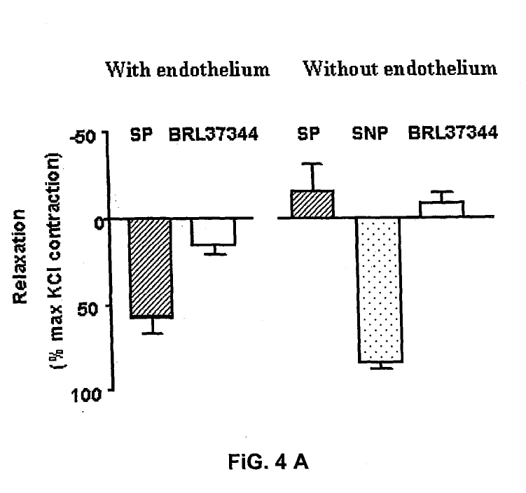

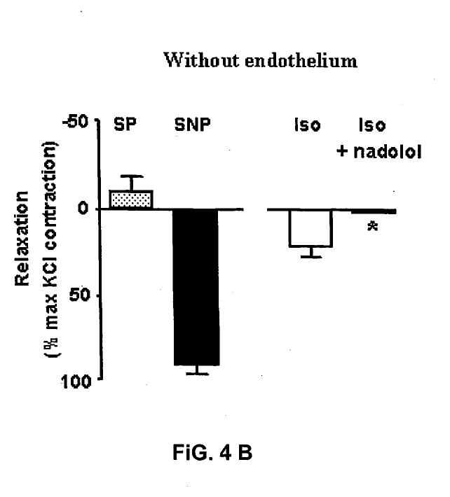

FIG. 4 shows a lack of beta-mediated relaxation in coronary microarteries devoid of endothelium-mediated response. A. Relaxations evoked by the beta₃-agonist BRL 37344 on KCl-preconstricted human coronary microarteries with functional (left) or destroyed endothelium (right), in comparison with the endothelium-specific agonist Substance P (n=4). BRL37344 failed to relax arteries without endothelium B. Relaxation evoked by the non-specific beta-agonist isoprenaline on de-endothelialized microarteries, as attested by the absence of relaxation to Substance P (100 nmol/L) despite full relaxation to sodium nitroprusside (100 μmol/L). The residual relaxation to Iso was abrogated by nadolol (n=4-7; P<0.05). Mean results (±sem) are expressed as % of the maximum KCl-evoked contraction.

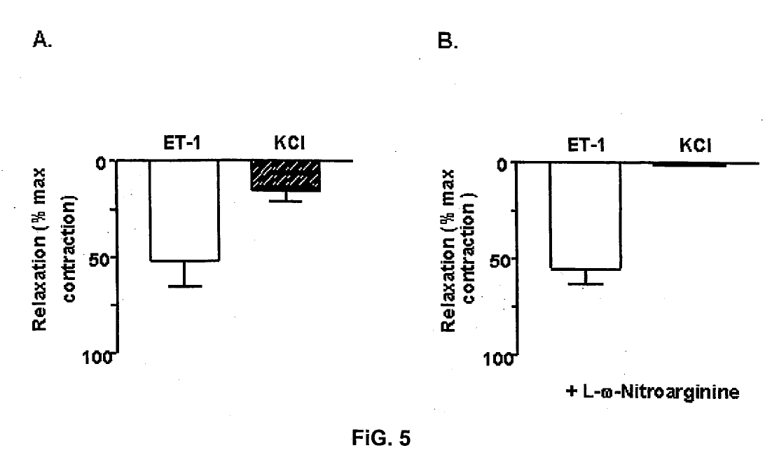

FIG. 5 illustrates that beta₃-mediated relaxation involves both NO and EDHF-like responses. Relaxations evoked by BRL37344 on human coronary microarteries precontracted with ET-1 (open bars) or KCl (black bars) in the absence or in the presence of the NOS-inhibitor L-ω-nitroarginine (100 μmol/L). Mean results (±sem) are expressed as % of the maximum ET-1 or KCl-evoked contraction (n=3-6). A. Precontraction with KCl eliminates the EDHF-like response and unveils residual NO-dependent relaxation; B. the NO-dependent relaxation is abrogated by NOS inhibition.

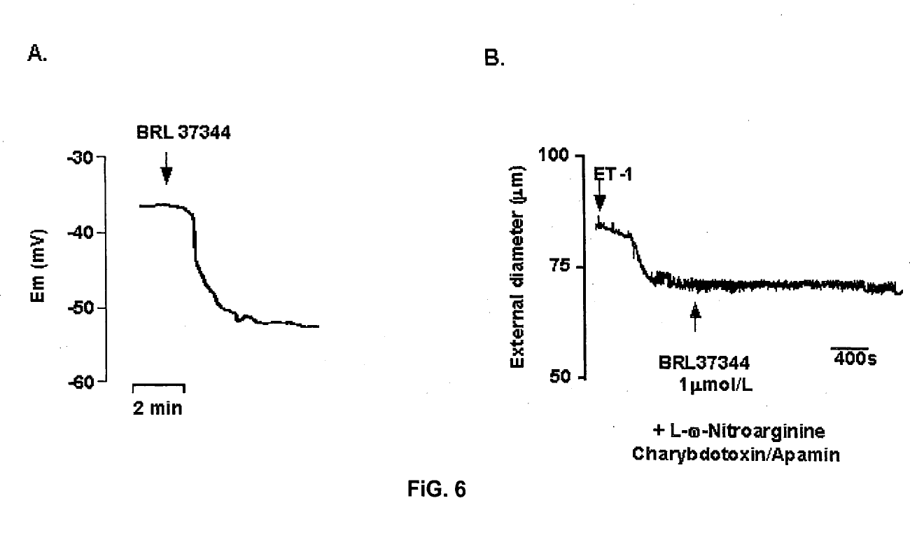

FIG. 6 shows the beta₃-agonist stimulation hyperpolarizes coronary microvessels and involvement of Ca²⁺-activated K⁺-channels. A. Typical recording showing the BRL37344-evoked hyperpolarization of smooth muscle cell membrane from isolated human coronary arteries. This tracing is representative of 5 similar experiments. B. Representative tracing of the contraction of a human coronary microarteriole with ET-1 after an incubation with the NOS inhibitor L-ω-nitroarginine, and K⁺ channels inhibitors, charybdotoxin and apamin (100 μmol/L each). Under these conditions no residual relaxation is observed in response to the beta₃-agonist BRL 37344. This tracing is representative of 3 similar experiments.

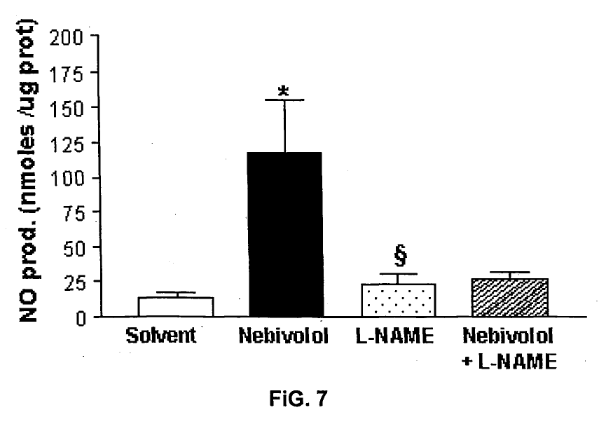

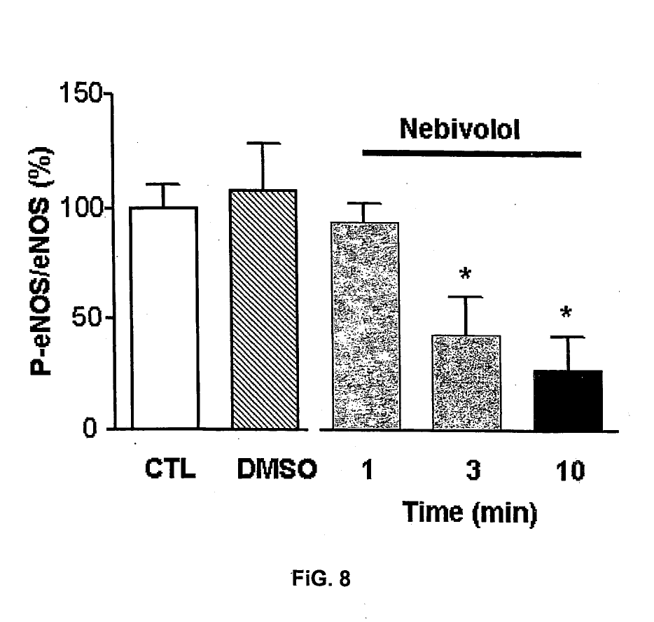

FIG. 7-11 illustrate several experiments showing that endothelial beta3-adrenoceptors mediate the NO-dependent vasorelaxation of human and rat coronary microvessels in response to the third-generation beta-blocker, nebivolol.

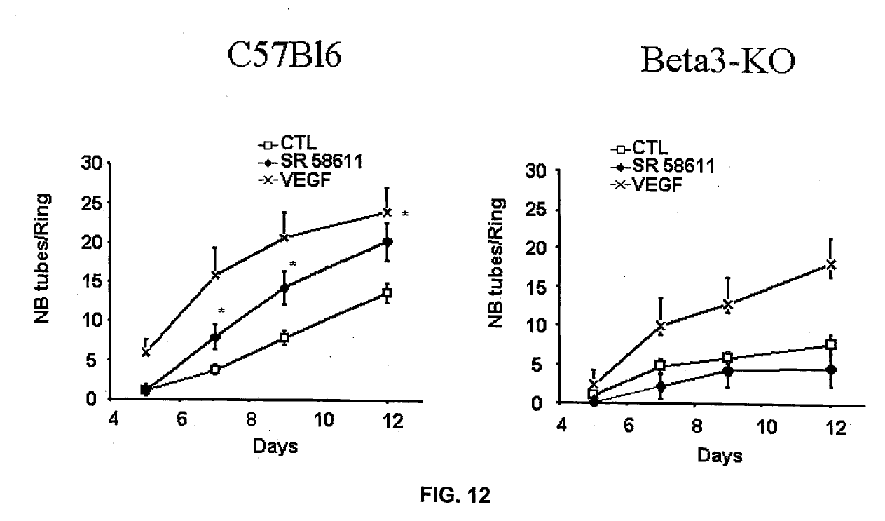

FIG. 12 illustrates pro-angiogenic effects of a beta3-adrenoceptor agonist (SR58611) on aortic rings obtained from C57B16 mice and mice genetically deficient in beta3-adrenoceptor (Beta-3 KO mice).

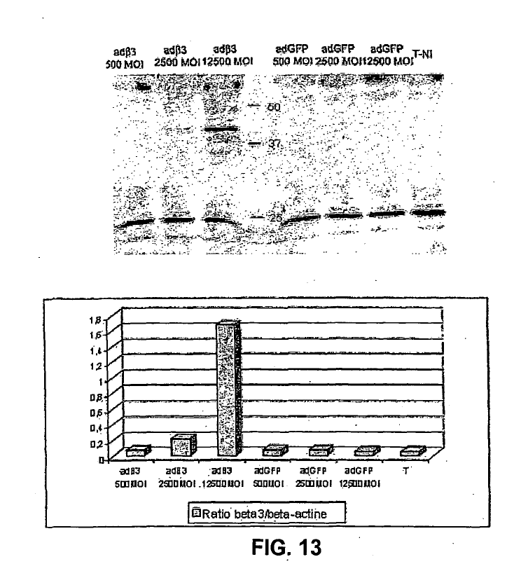

FIG. 13 illustrates the infection of human microvascular endothelial cells with an adenovirus encoding the human beta3 AR at different MOI (multiplicity of infections).

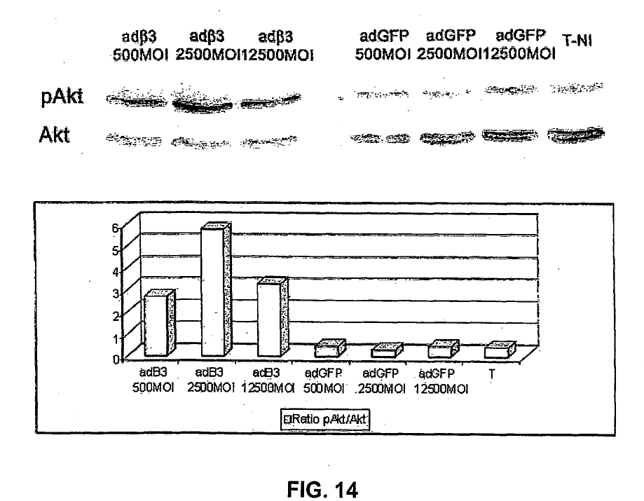

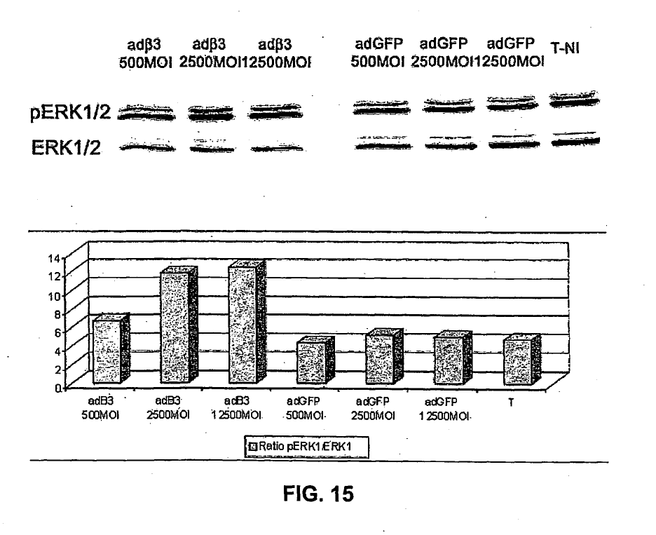

FIGS. 14 and 15 show that heterologously overexpressed human beta3 AR activates downstream signaling in human microvascular endothelial cells.

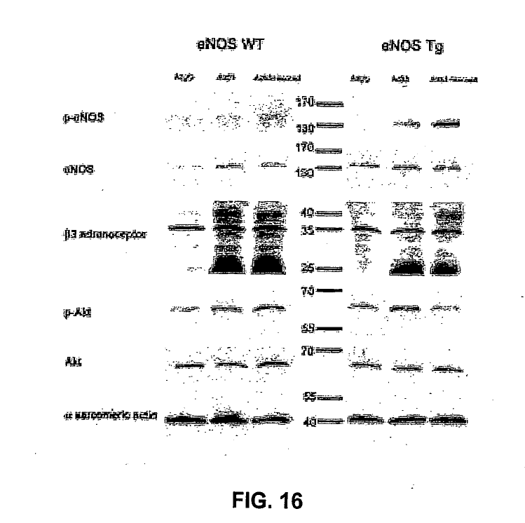

FIG. 16 shows that adenoviral expression of the human beta3 AR in cardiac myocytes from C57B16 control mice or mice overexpressing a cardiac-specific eNOS transgene induces activation of downstream signaling resulting in phosphorylation of Akt and eNOS.

### DETAILED DESCRIPTION OF THE INVENTION

The present invention is at least partly based on the finding that compounds having a beta-adrenoceptor agonistic effect according to the present invention show a strong and long-lasting coronary artery vasodilating action, peripheral blood vessel vasodilating action, and celebral blood vessel vasodilating action on mammals and are useful, for example, as preventive or curative agent for cardiovascular diseases such as diseases of circulatory systems, for example, coronary artery diseases, peripheral and cerebral circulatory disorders (e.g. cerebral infarction and transient cerebral ischemic attack), failing and ischemic cardiac diseases (e.g. angina pectoris and myocardial infarction), conditions related to metabolic syndrome, etc. . . .

Moreover, the applicant has shown that such compounds having a beta-adrenoceptor agonistic effect can be effectively combined with compounds having a beta1/beta2-adrenoceptor antagonistic effect, and that such combination provides particularly advantageous combination effects.

Beta-adrenergic receptors (beta-AR) are well known in the art as sites in the autonomic nervous system in which inhibitory responses occur when adrenergic agents, such as norepinephrine and epinephrine, are released. Beta-adrenergic receptors play a major role in modulating cardiac inotropic and chronotropic responses to catecholamines in numerous tissues. Activation of beta-receptors causes various physiological reactions, such as relaxation of the bronchial muscles and an increase in the rate and force of cardiac contraction. Agonists and antagonists of beta-adrenergic receptors have been identified in the prior art.

Beta3-AR belongs to a family of beta-adrenergic receptors which comprises three members: beta1-AR, beta2-AR and beta3-AR. Beta1-AR, beta2-AR antagonists have been extensively described in the prior art and are also commonly referred to as to “beta-blockers”. These antagonists block the activity of the beta1- and beta2-adrenergic receptors. The main activities of such antagonists include a reduction of the contraction force of the cardiac (heart) muscle, a decrease in the heart rhythm and frequency, and an anti-hypertension effect.

Beta3-AR is described as a metabolic receptor, as it mediates the beta-oxidation of fats. Agonists of beta3-AR have been described to be useful in the treatment of obesity and diabetes (type II). Since beta3-AR is expressed in tissues such as the gall bladder, the smooth muscle of the colon, bronchi, the prostate, etc. therapeutic applications of beta3-AR agonists in diseases affecting these tissues have also been described.

Beta3-AR has also been described to provide a negative inotropic effect, i.e. to induce decrease in the cardiac contraction. Beta3-AR antagonists have been reported to be suitable for blocking the negative inotropic effect of the beta3-AR and improve the cardiac function.

The present invention is based on the use of beta1-AR, beta2-AR, and beta3-AR agonists and antagonists in the treatment of various diseases as defined below, including arterial diseases such as coronary, peripheral and cerebral artery diseases, for treating ischemic and failing cardiac diseases and/or diseases related thereto, and/or for treating conditions related to metabolic syndrome.

### I. Definitions

The present invention is directed to methods, compositions and the use of beta-adrenergic receptors agonists and/or antagonists for treating and/or preventing cardiovascular diseases and various diseases that may result therein or that are related thereto.

As used herein, the term “cardiovascular diseases” refers to diseases which affect the heart (cardio) and/or the system of blood vessels (vascular). Cardiovascular diseases addressed herein may include but are not limited to arterial diseases, including coronary vascular diseases, cerebrovascular diseases, peripheral vascular disease, heart diseases such as ischemic heart diseases, failing hart diseases, conditions related to metabolic syndrome (Syndrome X), etc. . . .

The term “diseases related to cardiovascular diseases” as used herein refers to conditions that may cause and result in cardiovascular diseases, such as e.g. factors associated with metabolic syndrome (see below). This term also refers to diseases that are indirectly caused by diseases affecting the heart and vascular systems.

The term “metabolic syndrome” or “syndrome X” as used herein as synonyms and refer to various factors increasing the risk of developing cardiovascular diseases. such factors may include but are not limited to obesity, a decreased ability to process glucose (insulin resistance/and/or diabetes), dyslipidemia e.g. unhealthy lipid levels, low HDL and high LDL cholesterol levels, high triglyceride levels, abnormalities of blood clotting and hypertension, etc. . . .

The terms “arterial diseases” and “diseases of circulatory systems” are used herein as synonyms and refer to diseases wherein the circulatory systems, and in particular arteries are affected, resulting in a malfunctioning of the arteries and an inefficient blood flow. The term “arteries” is to be understood in its largest context, and includes all types of arteries such as e.g. coronary, peripheral or cerebral arteries. The term “diseases related to arterial diseases” as used herein refers to diseases which are indirectly caused by a dysfunction of the circulatory systems. Non-limitative examples of arterial diseases and diseases related thereto include for instance coronary artery diseases, peripheral artery diseases, cerebral artery diseases, etc. . . .

The “coronary arteries” as used herein, is meant to refer to any blood vessel which supplies blood to heart tissue of the subject and is meant to include native coronary arteries as well as those which have been grafted into the subject, for example, in an earlier coronary artery bypass procedure. The term “coronary artery diseases” as used herein refers to diseases directly affecting the coronary arteries, the blood vessels supplying the heart, causing narrowing and inadequate blood flow to the heart. The term “diseases related to coronary artery diseases” as used herein refers to diseases which are indirectly caused by diseases affecting the coronary arteries. Non-limitative examples of coronary artery diseases and diseases related thereto include atherosclerotic or non atherosclerotic disease of the coronary arteries, resulting e.g. from age, smoking, dyslipidemia (e.g. hypercholesterolemia and/or hypertriglyceridemia), insulin-dependent or insulin-independent diabetes mellitus, plurimetabolic syndrome, familial hereditary conditions, chronic high blood pressure, or a combination thereof, and all other conditions associated with dysfunction or loss of endothelial cells, including inflammatory, infectious, metabolic diseases, vascular disease associated with chronic dialysis and after surgery.

The “peripheral arteries” as defined herein includes any of the arteries outside the heart. In a preferred embodiment this term refers to arteries supplying blood the limbs. The term “peripheral arterial disease” as used herein refers to diseases directly affecting any of the arteries outside the heart causing narrowing and inadequate blood flow. The term “diseases related to peripheral artery diseases” as used herein refers to diseases which are indirectly caused by diseases affecting the peripheral blood vessels. Non-limitative examples of peripheral artery diseases and diseases related thereto include claudication, critical limb ischemia, chronic ischemic rest pain, ulcers, gangrene, etc.

The “cerebral arteries” are the vessels which bring the blood to the brain. The term “cerebral artery diseases” as used herein refers to diseases directly affecting the cerebral arteries, the blood vessels supplying the brain, causing narrowing and inadequate blood flow to the brain. The term “diseases related to cerebral artery diseases” as used herein refers to diseases which are indirectly caused by diseases affecting the cerebral arteries. Non-limitative examples of cerebral artery diseases and diseases related thereto include atherosclerosis, and congenital, traumatic, infectious, inflammatory, and other conditions.

The term “neo-angiogenesis” or “angiogenesis” are used herein as synonyms and refer to the phenomenon of the formation of new blood vessels. The term “angiogenesis-stimulating agent” as used herein refers to a compound improving neo-angiogenesis. The term “angiogenesis-related diseases” refers to diseases which are directly or indirectly caused by an ineffective angiogenesis process. An aberrant angiogenesis process is important in several pathologies in all parts of the body, involving all disciplines of medicine. Non-limitative examples of diseases associated with aberrant angiogenesis include for instance vascular diseases, ocular diseases, such as, age-related macular degeneration, diabetic retinopathy, corneal neovascularization, and stromal keratitis, retinal vasculitis, rheumatoid arthritis, certain types of cancers. Increase in angiogenesis would also be beneficial in a variety of ischemic cardiovascular diseases.

The term “ischemic cardiac diseases” refers to diseases based on ischemia, i.e. the deficiency of blood to a part of the body. Ischemic Heart Disease is a condition where the heart muscles do not receive proper blood supply. This is usually due to functional constriction or actual obstruction in the coronary vessels. IHD develops gradually and is mainly without pain or other symptoms in the initial stages.

“Failing heart disease” refers to a condition wherein the heart isn't pumping blood as well as it should. Heart failure is also called congestive heart failure. “Congestive” means fluid is building up in the body because the heart isn't pumping properly.

The term “diseases related to ischemic and failing cardiac diseases” refers to diseases which are indirectly caused by ischemia or failing heart conditions. Illustrative examples of ischemic and failing cardiac diseases include but are not limited to diseases such as angina pectoris, unstable angina, non Q-wave and transmural myocardial infarction, ischemic cardiomyopathy, hypertrophic cardiomyopathy, including resulting from chronic hypertension, aortic stenosis, aortic coarctation or other valvular or structural damage, idiopathic dilated cardiomyopathy, viral and auto-immune cardiomyopathy, post-transplantation cardiomyopathy and other diseases of the myocardium resulting from inflammatory, infectious, septic, metabolic toxic (including after treatment with anthracyclins) or structural cause, including through aging and cardiac valvular disease; in particular, cardiac diseases with diastolic dysfunction resulting from all of the above and any other cause. Illustrative examples of diseases related to ischemic and failing cardiac diseases include for instance endothelial dysfunction resulting from heart failure, including in cerebral and peripheral arterial trees.

The term “heart failure” refers to a condition in which a structural or functional deficiency of the cardiac muscle results in pump failure and the inability of the heart to supply sufficient blood flow to match the body's metabolic needs.

The term “diastolic heart failure” refers to a condition in which a structural or functional deficiency of the cardiac muscle affects more specifically the capacity of the ventricular chambers to relax between the contractions, thereby affecting their filling properties and proper pump functioning.

The term “metabolic deficiency in heart failure” refers to a condition in which metabolic remodeling, e.g. in the cardiac muscle, results in inappropriate use of metabolic substrates for energy production in the cardiac muscle thereby resulting in altered function.

The term “metabolic remodeling” refers to a condition in which structural, functional or biochemical alterations in the cardiac muscle resulting e.g. from an ischemic insult or the metabolic syndrome lead to changes in metabolic substrate utilization by the heart.

### II. Use of Beta-AR Agonists and Antagonists for the Preparation of a Medicament

In accordance with the present invention a novel activity of beta3-AR agonists has now been demonstrated. More in particular, it has now been shown that beta3-AR agonists provide a vascular effect on the beta3-AR receptor. It has been shown that beta3-AR is expressed in the endothelium of coronary micro-arteries of the human heart (see example below). In addition, it was also demonstrated that beta3-AR induce a relaxation (vasodilatation) of these coronary arteries (see example below). Beta3-AR agonists can also show a vasodilatation effect on peripheral or cerebral arteries. This novel effect of beta3-AR agonists makes these compounds particularly useful for treating diseases of circulatory systems.

In accordance with the present invention, it has further been demonstrated that a compound having beta3-adrenoceptor agonistic effect and exerting a vasodilating activity can be advantageously and effectively combined with other beta-AR agonists and/or antagonists, and in particular with compounds showing a beta1/beta2-adrenoceptor antagonistic effect. Such combination provides a double effect: an improvement of perfusion of the heart muscle (vasodilating mediated by the beta3-adrenoceptor agonistic activity) and a reduction of the contraction force of the cardiac muscle (mediated by the beta1/beta2-adrenoceptor antagonistic activity). Beta3 agonists, in addition to vasodilatation, also produce a negative inotropic effect on the human cardiac muscle, thereby acting synergistically with beta1-2 antagonists.

The term “compound having a beta3-adrenoceptor agonistic effect” refers to a compound showing a stimulating (agonistic) effect on the activity of a beta3-adrenoceptor. It is to be understood that this compound may be a beta3-adrenoceptor agonist per se or any other compound having a beta3-adrenoceptor agonist-like activity and thus showing a stimulating effect on the activity of a beta3-adrenoceptor. This stimulating effect results in a vasodilatation effect of blood vessels.

The term “compound having a beta1, beta2-adrenoceptor antagonistic effect” refers to a compound showing an inhibiting (antagonistic) effect on the activity of a beta1/beta2-adrenoceptor. It is to be understood that this compound may be a beta1/beta2-adrenoceptor antagonist per se or any other compound having a beta1/beta2-adrenoceptor antagonist-like activity and thus showing an inhibiting effect on the activity of a beta1/beta2-adrenoceptor.

The invention relates to the use of a compound having a beta3-adrenoceptor agonistic effect as a vasodilating agent. The term “vasodilating agent” as used herein refers to a compound improving the vasodilatation of blood vessels.

The present invention also further relates in another embodiment to the use of a compound having a beta3-adrenoceptor agonistic effect and a beta1/beta2-adrenoceptor antagonistic effect as vasodilating and beta-blocking agent. The term “vasodilating agent” is defined as above. The term “beta-blocking agent” refers to a compound that blocks the activity of beta-adrenoceptors, and by doing so, reduces the contraction force of the cardiac muscle.

### Cardiovascular Diseases and Diseases Related Thereto

In one further embodiment, the invention relates to the use of a compound having a beta3-adrenoceptor agonistic effect for the preparation of a medicament for treating and/or preventing cardiovascular diseases and diseases related thereto.

The invention may also relate to the use of a compound having a beta3-adrenoceptor agonistic effect and having a beta1/beta2-adrenoceptor antagonistic effect for the preparation of a medicament for treating and/or preventing cardiovascular diseases and diseases related thereto. The present invention may thus provide for the use of a single compound showing a double effect: i.e. a beta3-adrenoceptor agonistic effect and a beta1/beta2-adrenoceptor antagonistic effect.

In yet another embodiment, the invention relates to the use of one or more first compound(s) having a beta3-adrenoceptor agonistic effect and one or more second compound(s) having a beta1/beta2-adrenoceptor antagonistic effect for the preparation of a medicament for treating and/or preventing cardiovascular diseases and diseases related thereto, wherein said one or more first compound(s) and said one or more second compound(s) are used as combined preparation for simultaneous, separate or sequential use. The double effect may thus be obtained by using two or more different compounds. The applicant has demonstrated that certain compounds showing a beta1/beta2-adrenoceptor antagonistic activity may also show a beta3-adrenoceptor agonistic activity. Advantageously, the above-mentioned double effect may hereby be provided by one and the same compound. Non-limiting illustrative examples of such compounds include nebivolol, CGP12177 (a beta3 agonist with beta1/2 antagonistic properties), pindolol (a beta1/2 antagonist with beta3 agonistic properties), or a pharmacologically acceptable derivative thereof or any mixtures thereof. Alternatively, the above-mentioned double effect can be mediated by combining one or more first compound(s) having a beta3-adrenoceptor agonistic effect with one or more second compound(s) having a beta1/beta2-adrenoceptor antagonistic effect. According to the present invention, any compound having a beta1/beta2-adrenoceptors antagonistic activity may be combined with any beta3-adrenoceptors agonist activity. It is to be understood that these compounds may be beta3-adrenoceptor agonists and beta1/beta2-adrenoceptor antagonists per se or any other compound having a beta3-adrenoceptor agonist-like activity and beta1/beta2-adrenoceptor antagonist-like activity, respectively. Illustrative examples of suitable beta3-adrenoceptor agonists are provided below. Beta1/beta2-adrenoceptors antagonists are well known in the art and will not be discussed into detail herein. Non-limiting examples of suitable beta1/beta2-adrenoceptors antagonists for the present invention include acebutalol, atenolol, betaxolol, bisoprolol, carvedilol, celiprolol, esmolol, labetalol, metoprolol, nadolol, nebivolol, oxprenolol, pindolol, sotalol, propranolol, practolol, CPG 20712A, ICI 118551, timolol. Each type has one or more brand names. It is clear that also examples of beta3-adrenoceptor agonists and beta1/beta2-adrenoceptor antagonists, which are not provided herein, but which are known and available in the art, as well as beta3-adrenoceptor agonists and beta1/beta2-adrenoceptor antagonists that are under development or that will be developed in the future may be suitable for use in the present invention as well.

### Arterial Diseases

In a preferred embodiment, said treatment and/or prevention is further defined as treating and/or preventing arterial diseases and/or diseases related thereto. In another preferred embodiment, said treatment and/or prevention is further defined as treating and/or preventing coronary artery diseases and/or diseases related thereto. In yet another preferred embodiment, said treatment and/or prevention is further defined as treating and/or preventing peripheral artery diseases and/or diseases related thereto. In yet another embodiment, said treatment and/or prevention is further defined as treating and/or preventing cerebral artery diseases and/or diseases related thereto.

Therefore, in another embodiment, the invention relates to the use of a compound having a beta3-adrenoceptor agonistic effect for the preparation of a medicament for treating and/or preventing arterial diseases such as coronary artery diseases, peripheral artery diseases, and/or cerebral artery diseases, and/or diseases related thereto.

The invention may also relate to the use of a compound having a beta3-adrenoceptor agonistic effect and having a beta1/beta2-adrenoceptor antagonistic effect for the preparation of a medicament for treating and/or preventing arterial diseases such as coronary artery diseases, peripheral artery diseases, and/or cerebral artery diseases, and/or diseases related thereto.

In yet another embodiment, the invention relates to the use of one or more first compound(s) having a beta3-adrenoceptor agonistic effect and one or more second compound(s) having a beta1/beta2-adrenoceptor antagonistic effect for the preparation of a medicament for treating and/or preventing arterial diseases such as coronary artery diseases, peripheral artery diseases, and/or cerebral artery diseases, and/or diseases related thereto, wherein said one or more first compound(s) and said one or more second compound(s) are used as combined preparation for simultaneous, separate or sequential use. The double effect may thus be obtained by using two or more different compounds.

### Ischemic and Failing Cardiac Diseases

In another preferred embodiment, said treatment and/or prevention is further defined as treating and/or preventing ischemic and failing cardiac diseases and/or diseases related thereto. In a preferred embodiment, said failing cardiac disease comprises heart failure, and preferably diastolic heart failure.

Therefore, in another embodiment, the invention relates to the use of a compound having a beta3-adrenoceptor agonistic effect for the preparation of a medicament for treating and/or preventing ischemic and failing cardiac diseases and/or diseases related thereto, and preferably heart failure, and more preferably diastolic heart failure.

The invention may also relate to the use of a compound having a beta3-adrenoceptor agonistic effect and having a beta1/beta2-adrenoceptor antagonistic effect for the preparation of a medicament for treating and/or preventing ischemic and failing cardiac diseases and/or diseases related thereto, and preferably heart failure, and more preferably diastolic heart failure.

In yet another embodiment, the invention relates to the use of one or more first compound(s) having a beta3-adrenoceptor agonistic effect and one or more second compound(s) having a beta1/beta2-adrenoceptor antagonistic effect for the preparation of a medicament for treating and/or preventing ischemic and failing cardiac diseases and/or diseases related thereto, and preferably heart failure, and more preferably diastolic heart failure, wherein said one or more first compound(s) and said one or more second compound(s) are used as combined preparation for simultaneous, separate or sequential use.

### Syndrome X

In yet another embodiment, the treatment and/or prevention is further defined as treating and/or preventing one or more conditions related to metabolic syndrome (syndrome X). As mentioned above, metabolic syndrome is a collection of health risks that increase a person's chance of developing heart disease, stroke, and diabetes. Metabolic syndrome may be associated with conditions such as insulin resistance, accumulation of triglycerides, myocardial dysfunction, metabolic remodeling etc. In a preferred embodiment, the present invention provides the use of compounds as defined above for the preparation of a medicament, for treating and/or preventing metabolic remodeling and/or myocardial dysfunction, in particular in a state of insulin resistance during the metabolic syndrome.

Therefore, in another embodiment, the invention relates to the use of a compound having a beta3-adrenoceptor agonistic effect for the preparation of a medicament for treating and/or preventing one or more conditions related to metabolic syndrome, and preferably conditions such as metabolic remodeling and/or myocardial dysfunction.

The invention may also relate to the use of a compound having a beta3-adrenoceptor agonistic effect and having a beta1/beta2-adrenoceptor antagonistic effect for the preparation of a medicament for treating and/or preventing one or more conditions related to metabolic syndrome, and preferably conditions such as metabolic remodeling and/or myocardial dysfunction.

In yet another embodiment, the invention relates to the use of one or more first compound(s) having a beta3-adrenoceptor agonistic effect and one or more second compound(s) having a beta1/beta2-adrenoceptor antagonistic effect for the preparation of a medicament for treating and/or preventing one or more conditions related to metabolic syndrome, and preferably conditions such as metabolic remodeling and/or myocardial dysfunction, wherein said one or more first compound(s) and said one or more second compound(s) are used as combined preparation for simultaneous, separate or sequential use.

### Neo-Angiogenesis

In another aspect, it was demonstrated that a compound having a beta3-adrenoceptor agonistic effect stimulates the neo-angiogenesis process. It was also demonstrated that a compound having a beta3-adrenoceptor agonistic effect and a beta1/beta2-adrenoceptors antagonistic effect stimulates the neo-angiogenesis process.

The present invention therefore also relates to the use of a compound having a beta3-adrenoceptor agonistic effect as an angiogenesis-stimulating agent. The term “angiogenesis-stimulating agent” as used herein refers to a compound improving neo-angiogenesis. In another embodiment, the present invention also relates to the use of a compound having a beta3-adrenoceptor agonistic effect and a beta1/beta2-adrenoceptors antagonistic effect as an angiogenesis-stimulating agent. In yet another embodiment, the present invention also relates to the use of one or more first compound(s) having a beta3-adrenoceptor agonistic effect and one or more second compound(s) having a beta1/beta2-adrenoceptor antagonistic effect as an angiogenesis-stimulating agent.

In one embodiment, the invention further relates to the use of a compound having a beta3-adrenoceptor agonistic effect for the preparation of a medicament, wherein said compound further stimulates neo-angiogenesis. The present invention thus relates to the use of a compound having a beta3-adrenoceptor agonistic effect for the preparation of a medicament for stimulating neo-angiogenesis and/or for treating and/or preventing angiogenesis-related diseases.

In one embodiment, the use of a compound having a beta3-adrenoceptor agonistic effect and having a beta1/beta2-adrenoceptor antagonistic effect for the preparation of a medicament for stimulating neo-angiogenesis and/or for treating and/or preventing angiogenesis-related disease is encompassed.

The present invention further relates to the use of one or more first compound(s) having a beta3-adrenoceptor agonistic effect and one or more second compound(s) having a beta1/beta2-adrenoceptor antagonistic effect for the preparation of a medicament for stimulating neo-angiogenesis and/or for treating and/or preventing angiogenesis-related diseases, wherein said one or more first compound(s) and said one or more second compounds are used as combined preparation for simultaneous, separate or sequential use. The double effect may thus be obtained by using two or more different compounds.

### Other Effects

In a preferred embodiment, the invention relates to the use of a compound according to the invention having a beta3-adrenoceptor agonistic effect wherein said compound improves perfusion of the heart muscle. In another preferred embodiment, the invention relates to the use of said compound, wherein said compound improves perfusion of the heart muscle by improving coronary circulation. The term “coronary circulation” refers to the blood vessels that supply blood to, and remove blood from, the heart. In yet another embodiment, the invention relates to the use of a compound according to the invention having a beta3-adrenoceptor agonistic effect wherein said compound improves perfusion of the heart muscle by improving vasodilatation of coronary arteries. In another preferred embodiment, the invention relates to the use of compounds as defined above for the preparation of a medicament, wherein said compound(s) improve(s) perfusion of the heart muscle and reduce(s) the contraction force of the cardiac muscle. In another preferred embodiment, the invention relates to the use of compound(s) as defined above for the preparation of a medicament, wherein said compound(s) improve(s) perfusion of the heart muscle by improving coronary circulation. In yet another embodiment, the invention relates to the use of a compound for the preparation of a medicament, wherein said compound(s) improve(s) perfusion of the heart muscle by improving vasodilatation of coronary arteries.

In yet another embodiment, the invention relates to the use of one or more compounds as defined above, for the preparation of a medicament for protecting the heart against metabolic deficiency in heart failure.

In a further embodiment, the invention relates to the use of one or more compounds as defined above, according to the invention, wherein said compound has its effect via the modulation of NO production and/or the modulation of vessel hyperpolarization mechanisms. Preferably, said compound activates eNOS and the release of NO. In another preferred embodiment, said compound mediates vessel hyperpolarization through an EDHF-like (endothelium-derived hyperpolarizing factor(s)) response.

### Specific Compounds

In another embodiment, any compound having a beta3-adrenoceptor agonist activity; any compound having a beta3-adrenoceptor agonist activity and a beta1/beta2-adrenoceptor antagonistic activity, or any combination of at least one compound having a beta1/beta2-adrenoceptor antagonistic activity with at least one compound having a beta3-adrenoceptor agonist activity, may be used in the preparation of a medicament for treating and/or preventing one of the above-mentioned diseases as such. It will be understood from the present invention that a compound or compounds as defined above may also be used for the preparation of a medicament for simultaneously, separately or sequentially treating one or more of the above-mentioned diseases.

Preferably said compound having a beta3-adrenoceptor agonistic effect as defined herein is chosen from the group consisting of norepinephrine, epinephrine, BRL37344, SR 58611, CGP12177, nebivolol, isoproterenol, oxprenolol, carazolol, prenalterol, salbutamol, salmeterol, fenoterol, clenbuterol, +/−trimetoquinol, BRL28410, LY79771, LY362884, LY377604, CL 316243, CP331679, CP331684, AD9677, BMS196085, BMS187257, pindolol, (S)-(−)pindolol, ZD 7114, L755507, L749372, L750355, L757793, L760087, L764646, L766892, L770644, L771047, SM11044, SB251023, SB226552, SB229432, SB236923, SB246982, ICI201651, or a pharmacologically acceptable derivative thereof or any combinations thereof.

In a particularly preferred embodiment, the invention relates to the use of a compound according to the invention, wherein said compound is a specific or non specific beta3-adrenoceptor agonist. The difference between a (non specific) beta AR agonist and a specific beta AR agonist is the degree of selectivity, i.e. any compound having an agonistic activity on a beta-adrenoceptor (beta AR) can be called an agonist of that beta AR; if it only acts on a specific beta AR, it is a specific agonist of that beta AR.

A suitable example of a beta3-adrenoceptor agonist includes BRL37344. However, it is clear that also other examples of beta3-adrenoceptors agonists or of compounds having beta3-adrenoceptor agonist-like activity, which are not provided herein, but which are known and available in the art, as well as beta3-adrenoceptors agonists that are under development or that will be developed in the future may be suitable for use in the present invention as well.

Non-limiting examples of suitable synergetic combinations of compounds having beta3-adrenoceptor agonist activity with compounds having a beta1/beta2-adrenoceptor antagonistic activity include combinations of BRL37344 with nadolol and SR58611 with bisoprolol. In another example, suitable synergetic combinations may include a combination of any of the compounds selected from the group comprising norepinephrine, epinephrine, BRL37344, SR 58611, CGP12177, nebivolol, isoproterenol, oxprenolol, carazolol, prenalterol, salbutamol, salmeterol, fenoterol, clenbuterol, +/−trimetoquinol, BRL28410, LY79771, LY362884, LY377604, CL 316243, CP331679, CP331684, AD9677, BMS196085, BMS187257, pindolol, (S)-(−)pindolol, ZD 7114, L755507, L749372, L750355, L757793, L760087, L764646, L766892, L770644, L771047, SM11044, SB251023, SB226552, SB229432, SB236923, SB246982, ICI201651 or a pharmacologically acceptable derivative thereof, with any of the compounds selected from the group comprising acebutalol, atenolol, betaxolol, bisoprolol, carvedilol, celiprolol, esmolol, labetalol, metoprolol, nadolol, nebivolol, oxprenolol, pindolol, sotalol, propranolol, practolol, CPG 20712β, ICI 118551, timolol or a pharmacologically acceptable derivative thereof.

### III. Compositions

In a further embodiment, the invention relates to a composition containing a therapeutically effective amount of a first compound having a beta3-adrenoceptor agonistic effect and a therapeutically effective amount of a second compound having a beta1/beta2-adrenoceptor antagonistic effect as a combined preparation for simultaneous, separate or sequential use for treating any of the above-defined diseases.

In one embodiment, the invention relates to the use of a pharmacological composition containing a therapeutically effective amount of a first compound having a beta3-adrenoceptor agonistic effect and a therapeutically effective amount of a second compound having a beta1/beta2-adrenoceptor antagonistic effect as a combined preparation for simultaneous, separate or sequential use for treating and/or preventing cardiovascular diseases and diseases related thereto selected form the group comprising arterial diseases, ischemic and failing cardiac diseases, one or more conditions related to metabolic syndrome, and any diseases related thereto.

In another embodiment, the invention provides a pharmacological composition containing a therapeutically effective amount of a first compound having a beta3-adrenoceptor agonistic effect and a therapeutically effective amount of a second compound having a beta1/beta2-adrenoceptor antagonistic effect as a combined preparation for simultaneous, separate or sequential use for stimulating neo-angiogenesis and/or for treating and/or preventing angiogenesis-related diseases.

The compositions according to the invention do not represent a mere aggregate of known agents, but a new combination with the surprising, valuable property of combining a improved perfusion of the heart muscle with a reduction of the contraction force of the cardiac muscle. The present compositions provide improved, synergistic effects. Both components in the compositions can be mixed in a single preparation or can be prepared separately. When prepared separately, the components of the composition can be used simultaneous or sequentially, whereby the beta3-adrenoceptor agonist is applied before the beta1/beta2-adrenoceptor antagonist or vice versa.

The present invention also relates to a pharmacological composition comprising a therapeutically effective amount of a compound or compounds as defined in the present invention or a pharmacologically acceptable derivative thereof for treating and/or preventing arterial diseases, and in particular coronary, peripheral and/or cerebral artery diseases, and/or ischemic and failing cardiac diseases and/or diseases related thereto, and/or one or more conditions related to metabolic syndrome (Syndrome X).

The present invention further relates to a pharmacological composition comprising a therapeutically effective amount of a compound or compounds as defined in the present invention or a pharmacologically acceptable derivative thereof or a composition as defined herein for stimulating angiogenesis and/or for treating and/or preventing angiogenesis-related diseases.

The term “therapeutically effective amount” as used herein means that amount of active component(s) or pharmaceutical agent that elicits the biological or medicinal response in a tissue, system, animal or human that is being sought by a researcher, veterinarian, medical doctor or other clinician, which includes alleviation of the symptoms of the disease being treated. The therapeutic effective amount depends on the disease to be treated and the professional skill of a therapist.

The term “pharmacologically acceptable derivative thereof” as used herein includes but is not limited to “salts, solvates”.

For therapeutic use, the “salts” of the compounds according to the invention are those wherein the counter-ion is pharmaceutically or physiologically acceptable. The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such a sarginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

As used herein and unless otherwise stated, the term “solvate” includes any combination which may be formed by a compound of this invention with a suitable inorganic solvent (e.g. hydrates) or organic solvent, such as but not limited to alcohols, ketones, esters and the like.

The pharmaceutical preparations preferably contain 0.1 to 90% by weight of active components according to the invention. The pharmaceutical preparations can be prepared in a manner known per se to a person skilled in the art. One or more compounds according to the present invention are brought into a suitable administration form or dosage form which can then be used as a pharmaceutical in human medicine or veterinary medicine. A composition containing one or more compounds according to the present invention may be manufactured by, if necessary, by mixing an effective amount of the active ingredient with various pharmaceutical ingredients suitable for the final administration form, such as organic or inorganic carriers, excipients, binders, moistening agents, disintegrators, lubricants, and diluents and other additives suitable for parenteral administration, according to routine methods. When the composition containing one or more compounds according to the present invention is to be used for injection, the active ingredient can be sterilized with a suitable carrier.

In a preferred embodiment, the one or more compounds according to the present invention are suitable for being administered orally or parenterally and can be prepared as oral solid dosage form, oral liquid dosage form or injection, by using organic or inorganic carriers, excipients and other additives suitable for oral or parenteral administration, according to routine methods.

Oral solid dosage forms may include tablet, powder, fine particle, granule, capsule, pill and sustained-release type. In such solid dosage forms, one or more active substances are mixed with at least one inactive diluent, for example lactose, mannitol, glucose, micro-fine cellulose, starch, cornstarch, polyvinylpyrrolidone and metasilicate aluminate magnesium. According to routine methods, the composition, may satisfactorily contain additives other than inactive diluents, including for example binders such as hydroxypropyl cellulose and hydroxypropylmethyl cellulose (HPMC); lubricants such as magnesium stearate, polyethylene glycol, starch and talc; disintegrators such as fibrinogen calcium glycolate and cermellose calcium; stabilizers such as lactose; dissolution auxiliary agents such as glutamic acid or aspartic acid; plasticizers such as polyethylene glycol; and colorants such as titanium oxide, talc and yellow ferric oxide. If necessary, the resulting tablet or pill may satisfactorily be coated with sugar coating or films comprising substances solubilizable in stomach or intestine, such as sucrose, gelatin, agar, pectin, hydroxypropyl cellulose and hydroxypropylmethyl cellulose phthalate. The most preferable is an oral solid dosage form, which can be readily incorporated by patients by themselves and are convenient for storage and transfer.

Oral liquid dosage forms may include pharmaceutically acceptable emulsions, solutions, suspensions, syrups and elixirs and contains inactive diluents for general use, for example distilled water and ethanol. Other than inactive diluents, the composition may satisfactorily contain auxiliary agents such as moisturizers and suspending agents, sweeteners, flavor, fragrance, and preservatives.

Injections for intravenous, intra-muscular and subcutaneous injection may include sterile aqueous or non-aqueous solutions, suspensions and emulsions. The diluents for the aqueous solutions and suspensions include for example distilled water for injections and physiological saline. The diluents for the non-aqueous solutions and suspensions include for example propylene glycol, polyethylene glycol and vegetable oils such as olive oil, alcohols such as ethanol and polysorbate 80. Such composition may additionally contain auxiliary agents such as preservatives, moisturizers, dispersants, stabilizers (for example, lactose), and dissolution auxiliary agents (for example, glutamic acid, aspartic acid). These are sterilized by filtration through bacteria trapping filters or blending with sterilizing agents or under irradiation. These may satisfactorily be used to produce sterile solid compositions, which are dissolved in sterile water or sterile solvents for injections prior to use, and are then used.

The pharmaceutical compositions of this invention can be administered to humans or animals in dosage ranges specific for each component comprised in said compositions. The compound comprised in said composition can be administered together or separately. It will be understood, however, that specific dose level and frequency of dosage for any particular patient may be varied and will depend upon a variety of factors including the activity of the specific active component employed, the metabolic stability and length of action of that compound, the age, body weight, general health, sex, diet, mode and time of administration, rate of excretion, drug combination, the severity of the particular condition, and the host undergoing therapy.

In a preferred embodiment, the ratio of the first compound having a beta3-adrenoceptor agonistic effect to the second compound having a beta1/beta2-adrenoceptor antagonistic effect in the combined composition is appropriately determined, depending on the activity of the compounds and is such to obtain a therapeutic effect. Preferably, the dose of the first compound having a beta3-adrenoceptor agonistic effect and the dose of the second compound having a beta1/beta2-adrenoceptor antagonistic effect is appropriately determined, depending on each case, taking account of the patient's age, body weight, general health, sex, diet, time of administration, route of administration, rate of excretion, drug combination, type and extent of disease. The first compound having a beta3-adrenoceptor agonistic effect may usually be administered, in single or divided doses of about 0.001 mg/kg/day to about 10,000 mg/kg/d, with preferred doses being about 0.1 mg/kg/d to about 1,500 mg/kg/d, and more preferred levels being about 1 mg/kg/d to about 1000 mg/kg/d. The second compound having a beta1/beta2-adrenoceptor antagonistic effect may usually be administered, in single or divided doses of about 0.001 mg/kg/day to about 10,000 mg/kg/d, with preferred doses being about 0.1 mg/kg/d to about 1,500 mg/kg/d, and more preferred levels being about 1 mg/kg/d to about 1000 mg/kg/d.

### IV. Methods of Treatment

In a further embodiment, the invention relates to methods of treating and/or preventing cardiovascular diseases and diseases related thereto in a subject in the need thereof, including but not limited to arterial diseases, ischemic and failing cardiac diseases, including heart failure, conditions related to metabolic syndrome. The term “subject” as used herein may refer to a human or an animal.

In other embodiments, the present invention relates to the use of a one of more compounds according to the invention for the preparation of a medicament, wherein said compound(s) is (are) combined with a suitable excipient, for the treatment and or prevention of cardiovascular diseases and/or diseases related thereto, including but not limited to arterial diseases, ischemic and failing cardiac diseases, including heart failure, conditions related to metabolic syndrome. In an embodiment, the present invention relates to a method of treating and/or preventing cardiovascular diseases and diseases related thereto in a subject in the need thereof comprising administering (a) compound(s) as defined in the present invention, in a sufficient concentration able to exert a beta3-adrenoceptor agonistic effect. The invention also relates to a method of treating and/or preventing cardiovascular diseases and diseases related thereto in a subject in the need thereof comprising administering (a) compound(s) as defined in the present invention in a sufficient concentration able to exert a beta3-adrenoceptor agonistic and a beta1/beta2-adrenoceptor antagonistic effect. the invention also provides a method of treating and/or preventing cardiovascular diseases and diseases related thereto in a subject in the need thereof comprising administering a composition containing a therapeutically effective amount of a first compound having a beta3-adrenoceptor agonistic effect and a therapeutically effective amount of a second compound having a beta1/beta2-adrenoceptor antagonistic effect as a combined preparation for simultaneous, separate or sequential use.

In a preferred embodiment, the present invention relates to a method of treating and/or preventing an arterial disease, and preferably coronary, peripheral and cerebral artery diseases, and/or and diseases related thereto in a subject in the need thereof comprising administering (a) compound(s) as defined in the present invention, in a sufficient concentration able to exert a beta3-adrenoceptor agonistic effect. The invention also relates to a method of treating and/or preventing an arterial disease, and preferably coronary, peripheral and cerebral artery diseases, and/or diseases related thereto in a subject in the need thereof comprising administering (a) compound(s) as defined in the present invention in a sufficient concentration able to exert a beta3-adrenoceptor agonistic and a beta1/beta2-adrenoceptor antagonistic effect. The invention also provides a method of treating and/or preventing an arterial disease, and preferably coronary, peripheral and cerebral artery diseases, and/or diseases related thereto in a subject in the need thereof comprising administering a composition containing a therapeutically effective amount of a first compound having a beta3-adrenoceptor agonistic effect and a therapeutically effective amount of a second compound having a beta1/beta2-adrenoceptor antagonistic effect as a combined preparation for simultaneous, separate or sequential use.

In yet another preferred embodiment, the present invention relates to a method of treating and/or preventing ischemic and failing cardiac diseases and/or diseases related thereto, including heart failure, and preferably diastolic heart failure in a subject in the need thereof comprising administering (a) compound(s) as defined in the present invention, in a sufficient concentration able to exert a beta3-adrenoceptor agonistic effect. The invention also relates to a method of treating and/or preventing ischemic and failing cardiac diseases and/or diseases related thereto, including heart failure, and preferably diastolic heart failure in a subject in the need thereof comprising administering (a) compound(s) as defined in the present invention in a sufficient concentration able to exert a beta3-adrenoceptor agonistic and a beta1/beta2-adrenoceptor antagonistic effect. The invention also provides a method of treating and/or preventing ischemic and failing cardiac diseases and/or diseases related thereto, including heart failure, and preferably diastolic heart failure in a subject in the need thereof comprising administering a composition containing a therapeutically effective amount of a first compound having a beta3-adrenoceptor agonistic effect and a therapeutically effective amount of a second compound having a beta1/beta2-adrenoceptor antagonistic effect as a combined preparation for simultaneous, separate or sequential use.

In yet another preferred embodiment, the present invention relates to a method of treating and/or preventing one or more conditions related to metabolic syndrome, such as for example metabolic remodeling and/or myocardial dysfunction, in a subject in the need thereof comprising administering (a) compound(s) as defined in the present invention, in a sufficient concentration able to exert a beta3-adrenoceptor agonistic effect. The invention also relates to a method of treating and/or preventing one or more conditions related to metabolic syndrome, such as for example metabolic remodeling and/or myocardial dysfunction, in a subject in the need thereof comprising administering (a) compound(s) as defined in the present invention in a sufficient concentration able to exert a beta3 adrenoceptor agonistic and a beta1/beta2-adrenoceptor antagonistic effect. The invention also provides a method of treating and/or preventing one or more conditions related to metabolic syndrome, such as for example metabolic remodeling and/or myocardial dysfunction, in a subject in the need thereof comprising administering a composition containing a therapeutically effective amount of a first compound having a beta3-adrenoceptor agonistic effect and a therapeutically effective amount of a second compound having a beta1/beta2-adrenoceptor antagonistic effect as a combined preparation for simultaneous, separate or sequential use.

In yet another embodiment, the present invention relates to the use of a one of more compounds according to the invention for the preparation of a medicament, wherein said compound(s) is (are) combined with a suitable excipient, for the treatment and/or prevention of angiogenesis-related diseases. The invention relates in one embodiment to a method of treating and/or preventing angiogenesis-related diseases in a subject in the need thereof by administering (a) compound(s) as defined in the present invention in a sufficient concentration able to exert a beta3-adrenoceptor agonistic effect. The invention further relates to a method of treating and/or preventing angiogenesis-related diseases in a subject in the need thereof by administering (a) compound(s) as defined in the present invention in a sufficient concentration able to exert a beta3-adrenoceptor agonistic and a beta1/beta2-adrenoceptor antagonistic effect. The invention also provides a method of treating and/or preventing angiogenesis-related diseases in a subject in the need thereof comprising administering a composition containing a therapeutically effective amount of a first compound having a beta3-adrenoceptor agonistic effect and a therapeutically effective amount of a second compound having a beta1/beta2-adrenoceptor antagonistic effect as a combined preparation for simultaneous, separate or sequential use.

In still another embodiment, the present invention relates to the use of a one of more compounds according to the invention for the preparation of a medicament, wherein said compound(s) is (are) combined with a suitable excipient, for the stimulation of neo-angiogenesis. The invention relates in one embodiment to a method of stimulating neo-angiogenesis in a subject in the need thereof by administering (a) compound(s) as defined in the present invention in a sufficient concentration able to exert a beta3-adrenoceptor agonistic effect. The invention further relates to a method of stimulating neo-angiogenesis in a subject in the need thereof by administering (a) compound(s) as defined in the present invention in a sufficient concentration able to exert a beta3-adrenoceptor agonistic and a beta1/beta2-adrenoceptor antagonistic effect. The invention also provides a method of stimulating neo-angiogenesis in a subject in the need thereof comprising administering a composition containing a therapeutically effective amount of a first compound having a beta3-adrenoceptor agonistic effect and a therapeutically effective amount of a second compound having a beta1/beta2-adrenoceptor antagonistic effect as a combined preparation for simultaneous, separate or sequential use.

The following examples serve to more fully describe the manner of using the above-described invention. It is understood that these examples in no way serve to limit the true scope of this invention, but rather are presented for illustrative purposes.

### EXAMPLES

### Example 1

In the present study, we therefore examined the putative expression and functional role of beta3-adrenoceptors in human coronary arterioles. We identified transcripts and proteins specific for beta3-adrenoceptors in the endothelium of these vessels, and their activation mediated an endothelium-dependent relaxation that involves both NO and vessel hyperpolarization.

Methods and results: We examined the expression and functional role of beta₃-adrenoceptors in human coronary microarteries (HCμa) and their coupling to vasodilating nitric oxide (NO) and/or hyperpolarization mechanisms. Beta3-adrenoceptor mRNA and protein expression was demonstrated in extracts of HCμa. Immunohistochemical analysis revealed their exclusive localization in the endothelium, with no staining of vascular smooth muscle. In contractility experiments using video-microscopy, the non-specific beta-agonist, isoprenaline and the beta₃-preferential agonist, BRL37344 evoked a ˜50% relaxation of endothelin-1-preconstricted HCμa. Relaxations were blocked by the beta₋₁₋₂₋₃ adrenoceptor antagonist bupranolol but insensitive to the beta₁/beta₂ adrenoceptor antagonist, nadolol, confirming a beta₃-adrenoceptor mediated pathway. Relaxation to BRL37344 was absent in HCμa devoid of functional endothelium. When HCμa were precontracted with KCl (thereby preventing vessel hyperpolarization), the relaxation to BRL37344 was reduced to 15.5%, and totally abrogated by the NO synthase inhibitor, L-w-nitroarginine, confirming the participation of a NOS-mediated relaxation. The NOS-independent relaxation was completely inhibited by the K_{Ca2+} channels inhibitors, apamin and charybdotoxin, consistent with an additional EDHF-like response. Accordingly, membrane potential recordings demonstrated vessel hyperpolarization in response to beta3-adrenoceptor stimulation.

Conclusion: beta₃-adrenoceptors are expressed in the endothelium of human coronary resistance arteries, and mediate adrenergic vasodilatation through both NO and vessel hyperpolarization.

### Introduction

In non-vascular smooth muscle, such as in the colon or stomach, beta-adrenergic agonists elicit a relaxation through activation of an <<atypical>> beta-adrenoceptor, more recently identified as the beta3-adrenoceptor(⁷). Since its molecular identification(⁸), the distribution and functional role of this receptor has been extended from the regulation of lipolysis in fat tissue(⁹) to modulation of cardiac contraction(¹⁰), including in human ventricle(¹¹).

Endothelial cells modulate the vascular tone through both shear-stress and agonist-evoked release of vasorelaxants such as nitric oxide, prostacyclin and (still incompletely characterized) endothelium-derived hyperpolarizing factor(s), or EDHF. The functional importance of the latter was suggested to be inversely correlated with vessel diameter(^{17;18}), and to be more prominent in circumstances of impaired NO-mediated vasorelaxation, such as associated with risk factors for atherosclerosis and ischemic diseases (^{19;20}) An EDHF-mediated relaxation was observed in human resistance (including coronary) arteries in response to arachidonic acid (²¹) and adrenomedulin(²²).

### Methods.

### Tissue Collection

Human right atrial and left ventricular tissue specimens obtained from patients undergoing cardiac surgery were placed in physiological saline solution (PSS) containing (mmol/L): NaCl, 120; KCl, 5.9; NaHCO₃, 25; dextrose, 17.5; CaCl₂, 2.5; MgCl₂, 1.2; NaH₂PO₄, 1.2 (pH 7.4) maintained at 0-4° C., and carefully dissected to isolate microarteries. Coronary microarteries (70-170 μm diameter, 1-2 mm length), rapidly cleaned of adherent connective tissue were either kept at −80° C. until homogenization or used for functional experiments.

Reverse-Transcription and Polymerase Chain Reaction for mRNA Amplification.

Human coronary microvessels were homogenized in GTC buffer (Tripure, Roche). After reverse transcription, TaqMan® PCR was performed as described previously (²³). The Ct (threshold cycle) was defined as the cycle number at which the reporter fluorescence generated by cleavage of the probe crossed a fixed threshold above baseline. In the absence of template, its value amounted to 39.9±0.08 (n=3), indicating negligible background fluorescence from the probes. Specific primer and TaqMan® probe sequences for the human beta3 adrenoceptor and human NOS3 (endothelial NOS) were designed as previously published (23;24)

Protein immunoblotting.

Microdissected vessels pooled from 6-9 atrial or ventricular specimens were grinded in liquid nitrogen. Extracts were homogenized in 60 μL of buffer (mmol/L: Tris.HCl, 20 pH:7.4; EDTA, 2.5; NaCl, 100; NaF, 10; Na₃VO₄, 1; NaDeoxycholate, 1%; SDS, 0.1%; Triton X100, 1% and protease inhibitors cocktail). Protein samples were subjected to electrophoresis, transferred onto PVDF membranes and immunoblotted as described previously(¹¹), with antibodies directed against human beta₃-adrenoceptors and eNOS. Immunostaining of beta₃-adrenoceptors.

Pieces of atrial appendages were embedded in TissueTek OCT compound (Milesinc., Elchart, Ind.) and snap-frozen in precooled isopentane at −80° C. Prewashed fixed cryosections (5 μm; 3.5% formaldehyde) were incubated with monoclonal anti-human beta₃-adrenoceptor antibodies (1/200 in PBS with 1% BSA), then rinsed (PBS 0.1% BSA) and incubated with secondary polyclonal rabbit IgG (1/200) coupled to peroxidase. After washing, sections were counterstained with Mayer's haematoxylin and mounted for optical microscopy. Negative controls were performed in parallel where the primary antibody was omitted.

### Videomotion Analysis of Vessel Contraction.

Vessels were cannulated with dual glass micropipettes and secured with 10-0 nylon monofilament sutures in a Plexiglas isolated organ chamber circulated with oxygenated PSS (37° C.) and placed on an inverted microscope (Axiovert S100, Zeiss, Germany) connected to a CCD camera. Microvessels were pressurized with a PSS-filled burette manometer at 60 mmHg (a pressure chosen to avoid stretch-dependent effects typically manifested at higher pressures) in a no-flow state. Digitized imaging (IONOPTIX Corporation, Milton, Mass.) allowed continuous monitoring of vessel external diameter. All experiments were carried out in the presence of cyclooxygenase inhibitor (indomethacin, 10 μmol/L). After 30-45 min equilibration, vessels were contracted with high KCl solution (PSS, 50 mmol/L KCl replacing NaCl stoechiometrically). At the maximum of contraction, vasorelaxation with Substance P (100 nmol/L) was systematically tested to assess the presence of a functional endothelium. In some experiments, the endothelium was selectively destroyed by an air bolus. All reagents were added in the bathing solution.

### Measurement of Vessel Membrane Potential.

Microvessels were mounted in an organ bath continuously circulated (6 mL/min) with oxygenated PSS at 37° C. All experiments were performed in the presence of a NO synthase inhibitor (L-w-nitroarginine, 100 μmol/L) and indomethacin (10 μmol/L). After 60 minutes of equilibration, measurement of the smooth muscle membrane potential (Em) was made with a glass microelectrode (Clark, Electromedical instruments, type GC 120F-15) filled with 1.5M KCl. The input resistance of the microelectrodes varied between 50 and 80 MΩ. Differences in electric potential were measured with a Dagan amplifier (8100, Minneapolis, Minn., U.S.A.) and recorded. Criteria for a successful impalement were (1) an abrupt drop in voltage on entry of microelectrode into the cell, (2) stable membrane potential for at least 2 min, and (3) a sharp return to zero on withdrawal of the electrode.

### Statistics.

All results are expressed as mean ±sem. Statistical comparisons were performed by use of Student's t test or one-way ANOVA where appropriate. P values <0.05 were considered significant.

### Results

### Patient Population.

Clinical patient characteristics are summarized in Table 1. Samples were obtained from patients undergoing cardiac transplantation (n=4) or other cardiac surgical procedures (n=60). Most patients suffered from ischemic cardiac diseases (76%). All were treated with a variable combination of drugs as detailed in Table 1. All tissue collections have been approved by the local Ethics Committee.

### Endothelial-Restricted Expression of Beta₃-Adrenoceptors in Human Coronary Arterioles.

An analysis of 3-adrenoceptor and NOS3 mRNAs was performed by RT-PCR using dissected arterioles from right auricular appendages. Amplimers for both transcripts were detected from 3 different preparations, with a mean Ct of 34.0±0.15 (P<0.0001 vs. background; n=3) for beta₃-adrenoceptors and 36.0±1.1 for NOS3 (P=0.022 vs. background; n=3). By comparison, the highly expressed housekeeping gene GAPDH generated detectable signals at a mean Ct of 26.0±0.26 when amplified from the same cDNAs.

FIG. 1 illustrates the identification of specific proteins with immunoaffinity for anti-human beta₃-adrenoceptor antibodies both in Western blotting (1A) and immunohistochemistry (1B). Bands corresponding to beta₃-adrenoceptors and eNOS were detected both in whole cardiac extracts from left ventricle and atria and in extracts of arterioles microdissected from the same tissues. In both cases, signals for beta₃-adrenoceptors and eNOS are more intense in atrial versus ventricular extracts. To identify the specific cell type(s) expressing beta₃-adrenoceptors, immunohistochemical analysis was carried out in sections of human atrial myocardium. A positive staining was observed in cardiomyocytes, as previously described by us(¹¹). In addition, sections of arterioles stained positively (1B, upper left). Higher magnification revealed exclusive staining of endothelial cells of microarteries. No staining was observed in capillary endothelial cells (closely apposed to cardiomyocytes) or vascular smooth muscle cells (1B, lower).

beta₃-Adrenoceptors Mediate a Relaxation of Human Coronary Arterioles.

To assess the function of beta₃-adrenoceptor signaling in the same vessels, variations of external diameter of pre-constricted, pressurized human coronary microarterioles were studied by videomicroscopy with different beta-adrenoceptor agonists. As illustrated by the typical experiment presented on FIG. 2A, in vessels with an intact endothelium, the non-specific beta-agonist, isoproterenol relaxed endothelin-1 (ET-1) pre-constricted microarterioles by half. This relaxation was unaffected upon pre-treatment with the beta₁₂ antagonist, nadolol, thereby ruling out a beta₁₂-adrenoceptors mediated effect, but fully abrogated with the beta₁₋₂₋₃ antagonist, bupranolol, suggesting a beta₃-adrenoceptor mediated effect (FIG. 2B). In support of the latter, the preferential beta₃-adrenoceptor agonist, BRL 37344 produced a dose-dependent relaxation of the same amplitude (FIG. 2C), the maximum relaxation to BRL 37344 amounting to 52.3±13.2% of the ET-1 contraction (n=6). This relaxation was also resistant to nadolol (54.9±16.8%, n=5), but fully abrogated by bupranolol (n=4; FIG. 2D).

In coronary microarterioles with an intact endothelium pretreated with nadolol and phentolamine (combining alpha₁₂ and beta₁₂ adrenoceptors blockade), norepinephrine (1 micromol/l) also evoked a relaxation amounting to 41.4±7% (n=3) of ET-1 contraction (FIG. 3).

Of note, the relaxation to BRL37344 was not observed in vessels that failed to relax to the endothelium-specific agonist Substance P (not shown) or in which the endothelium was selectively destroyed, despite their full relaxation with sodium nitroprusside (a NO donor acting on the smooth muscle), confirming that the beta₃-adrenoceptor response is dependent on a functional endothelium (FIG. 4A). Conversely, these de-endothelialized vessels exhibited a residual relaxation to isoproterenol (21.0±6.3%, n=6), that was inhibited by nadolol (1.4±0.5%, n=4) (FIG. 4B). This identified an additional endothelium-independent, beta1-2 adrenergic response on the vascular smooth muscle.

The beta₃-Adrenoceptor-Mediated Relaxation Involves both NO and an Endothelium-Derived Hyperpolarizing Factor.

To characterize the endothelial mediator for the beta₃-adrenoceptor relaxation, the effect of BRL37344 was first compared in vessels pre-constricted with ET-1 or a high (50 mmol/L) KCl solution. The latter is known to depolarize the vessel membrane and prevent the relaxing effect of a (EDHF-like) hyperpolarizing factor. As shown in FIG. 5A, although BRL 37344 relaxed vessels preconstricted with both ET-1 and KCl, its relaxing effect was substantially reduced in vessels contracted with KCl (ET-1:52.3±13.2% (n=6) versus KCl: 15.5±5.3%, n=4), suggesting the participation of an EDHF-like response.

To determine the involvement of nitric oxide production in the KCl-resistant relaxation, similar experiments were performed in vessels pre-incubated with the NO synthase inhibitor, L-w-nitroarginine. NO synthase inhibition abrogated the residual relaxation with BRL37344 in KCl-preconstricted vessels, confirming NOS involvement in the beta₃-adrenoceptor response. Of note, the relaxing effect of BRL37344 on ET-1 preconstricted vessels was unaffected by NOS inhibition, suggesting compensation by the EDHF-like response (FIG. 5B).

Two additional approaches were used to confirm the involvement of an EDHF-like response in the NO-independent beta₃-adrenoceptor mediated relaxation. First, we tested the effect of BRL37344 on the membrane potential of human arterioles mounted in the same conditions as for the relaxation assays. As illustrated in FIG. 6A, acute application of BRL37344 in the presence of NO synthase and cyclooxygenase inhibition (to rule out confounding effects of NO and prostanoids) resulted in a significant hyperpolarization. Second, the sensitivity of the BRL37344-mediated relaxation to the calcium-activated K⁺ channels inhibitors, charybdotoxin and apamin was tested in similar vessels under video-microscopy. As shown in FIG. 6B, these inhibitors fully abrogated the residual relaxation, further confirming its mediation through an EDHF pathway.

### DISCUSSION

We characterized a novel pathway for the adrenergic vasorelaxation of human coronary microarteries through activation of beta₃-adrenoceptors on endothelial cells. This is distinct from the vasodilation that follows activation of adenylyl cyclase and increases in cAMP, most often ascribed to beta₂ (and perhaps beta 1) adrenoreceptor activation in vascular smooth muscle cells. Indeed, our functional experiments with non-specific beta-adrenergic (as well as beta₃-preferential) agonists demonstrate a vasorelaxation of human coronary microarteries with slow kinetics, similar to the smooth muscle relaxation attributed to the activation of <<atypical >> beta-adrenoceptors in other tissues. The fact that this vasorelaxation was insensitive to nadolol (a beta₁₂ adrenoceptor antagonist) and abrogated by bupranolol (a full beta-antagonist) strongly supported a beta₃-adrenoceptor-mediated response.

Accordingly, we provide evidence for the expression of mRNA and proteins of beta₃-adrenoceptors in extracts of dissected cardiac microarterioles. We had successfully used the same molecular approaches to identify and quantitate human beta₃-adrenoceptors in whole human myocardium(¹¹). Using immunohistochemistry, we show that, in addition to cardiomyocytes, beta₃-adrenoceptor expression is restricted to the endothelium of microarteries, with no staining of vascular smooth muscle. This is consistent with similar endothelium-restricted expression in rat aorta(¹⁶). It would also account for the lack of beta₃-adrenoceptor-mediated relaxation in vessels with dysfunctional or destructed endothelium in which we failed to obtain a typical endothelial-mediated relaxation with Substance P. Likewise, such response may have been undetected in previous studies using HCμa from patients with end-stage heart failure and endothelial dysfunction, leaving only a residual smooth muscle-mediated beta₂-adrenergic response (³).

Aside from prostanoids, nitric oxide and endothelium-derived hyperpolarizing factor(s) account for the prototypical endothelium-mediated vasorelaxation. Consistent with the expression of eNOS in our vessels, we found the beta₃-adrenoceptor relaxation to be partly mediated through NO production. This was evidenced by its complete abrogation by NOS inhibition under circumstances when both prostanoids and EDHF are inoperative (i.e. after cycloxygenase inhibition and preconstriction with high KCl, respectively). This also recapitulates our previous demonstration of a functional coupling of beta₃-adrenoceptor agonists to NO production in whole human ventricular muscle through G-alpha-i proteins(¹¹). However, NOS inhibition had little (if any) effect on the vasorelaxation of vessels preconstricted with ET-1. This cannot be explained by incomplete NOS inhibition, since similar treatment with L-w-nitroarginine did abrogate the endothelium-dependent relaxation to Substance P in the same vessels preconstricted with KCl (not shown). This strongly indicated the involvement of an alternative, EDHF-like response.

Although the precise nature of EDHF is still elusive, a consensus view is that this (these) factor(s) released from endothelial cells produce a hyperpolarization leading to vascular muscle relaxation through activation of calcium-dependent K⁺ channels(²⁵). Our results in vessels constricted with high KCl solution (which modifies the electrochemical gradient for K⁺ ions, thereby preventing hyperpolarization) are in agreement with the participation of an EDHF. In addition, we directly demonstrated vessel hyperpolarization in response to beta₃-adrenoceptor agonists and the abrogation of beta₃-adrenoceptor-mediated relaxation after vessel pre-treatment with the K⁺ channels inhibitors, charybdotoxin and apamin, two signatures of an EDHF response. Of note, the apparent insensitivity of the beta₃ relaxation to NOS inhibition in vessels preconstricted with ET-1 would suggest that this EDHF response fully compensates for the absence of NO. Indeed, previous reports have suggested EDHF to act as a back-up relaxing mechanism in circumstances of endothelial NO-dependent dysfunction (^{18;20;26}).

### Pathophysiological Implications

Our demonstration of a functional beta₃-adrenoceptor vasorelaxation mediated in part by EDHF in human coronary resistance arteries may have a major bearing on the understanding of the regulation of coronary perfusion in circumstances such as dyslipidemia, diabetes and atherosclerosis, all associated with decreased NO production and/or bioavailability. Indeed, previous work in human arteries suggested EDHF to be preserved despite the presence of risk factors for atherosclerosis (as is the case in the present study using arterioles mostly from ischemic patients)(^{18;27}). Notably, the natural catecholamine, norepinephrine, elicited similar beta₃-adrenoceptor vasorelaxation, extending the relevance of our paradigm. Given the relative resistance of beta₃-adrenoceptors to homologous desensitization(²⁸), their activation of such back-up relaxation are particularly appropriate in circumstances of increased adrenergic tone, such as ischemia or heart failure, to preserve myocardial perfusion.

TABLE 1
Patient data, clinical diagnosis and treatment.

| Patient data | |
|---|---|
| Age range, y | 12-83 |
| Average, y | 57 |
| Female, n | 10 |
| Male, n | 54 |
| Total, n | 64 |
| Clinical diagnosis | |
| Aortic valve replacement or repair, Ross intervention*, n | 12 |
| Mitral valve repair or replacement, n | 3 |
| Coronary artery bypass*, n | 48 |
| Cardiac transplant, n | 4 |
| Ischemics versus non-ischemics cardiac diseases, n | 49 versus 15 |
| Treatment regimes | |
| beta-blockers | 36/64 |
| Calcium antagonists | 10/64 |
| ACE inhibitors/Angiotensin receptor blockers | 27/64 |
| Antiarrhythmic | 2/64 |
| Digitalis | 1/64 |
| Diuretics | 13/64 |

In conclusion, this example illustrates that the a) beta3-AR receptor is expressed in the endothelium of coronary micro-arteries of the human heart; b) the beta3-AR receptor mediates a relaxation of these coronary arteries in response to beta3-AR agonists and c) the mechanism implicates NO and a endothelium-dependent hyperpolarization factor.

Aside from controlling cardiac contraction force, catecholamines dilate coronary arteries. Accordingly, we showed that beta₃-adrenoceptors are expressed in the endothelium of human coronary resistance arteries. Ex vivo, these vessels vasodilate when exposed to beta₃-specific or non-specific adrenoceptor agonists, a relaxation resistant to beta₁₋₂-adrenoceptor blockers. This relaxation involved both nitric oxide and vessel hyperpolarization through potassium channels. This novel coronary vasodilatory pathway opens new avenues for the treatment of ischemic heart disease.

### Example 2

This example demonstrates that endothelial beta3-adrenoceptors mediate the NO-dependent vasorelaxation of human and rat coronary microvessels in response to the third-generation beta-blocker, nebivolol.

The therapeutic effects of non-specific beta-blocking agents is often limited by vasoconstriction. Nebivolol is a selective antagonist at the beta1-adrenoceptor (AR) that releases the vasodilator, Nitric Oxide (NO) through incompletely characterized mechanisms. Endothelial beta3-adrenoceptors were identified in human coronary resistance arteries. Here it is demonstrated that nebivolol exerts a partial agonist effect on these beta3-AR to mediate NO— and endothelial-dependent relaxation.

Human and rat cardiac coronary resistance microarteries (70-170μ diameter) were mounted in dual glass micropipettes chambers in no-flow state and constant pressure for vasomotion analysis by videomicroscopy. In addition, calcium transients and NO release were measured in cultured endothelial cells with an amperometric electrode and Fura-2 fluorescence, respectively. Phosphorylation of eNOS was measured in the same cells with phospho-specific antibodies.

In endothelial cells, nebivolol (1-10 μM) increased NO release to 117+38 nmoles/μg prot (at 10 μM; n=3) in a L-NAME-inhibitable fashion (26.5±4.3 nmoles/μg prot; P<0.05). In parallel, Threonine 495 on eNOS was dephosphorylated (n=3; p<0.05) and Ca fluorescence increased by 91.8±23.7% (n=4-11). Pre-treatment with the beta1-2 antagonist, nadolol, had no effect on the Ca signal increase with nebivolol, whereas bupranolol, a combined beta1-2-3 antagonist, significantly blunted the effect (P<0.05). Nebivolol evoked a dose-dependent relaxation of rat microvessels (max 86±6% of PGF2alpha pre-contracted tone, at 10 μM) that was sensitive to NOS inhibition, unaffected by nadolol, but prevented by bupranolol (P<0.05; n=3-8). Importantly, the relaxation to nebivolol was blunted in microvessels from mice genetically deficient in beta3-AR. In human coronary microvessels, nebivolol (10 μM) also induced a relaxation (max 71±5% of ET-1 pre-contracted tone) that was dependent on a functional endothelium, insensitive to nadolol and reproduced with the beta3-preferential agonist, BRL37344 (all p<0.05). In human microvessels, beta3-AR expression was identified in endothelial cells by immunohistochemistry and laser capture/PCR.

As shown on FIG. 7, In Baecs, Nebivolol (10 μM) evoked a release of NO that is sensitive to L-Name.

As shown on FIG. 8, Thr495 on eNOS underwent a time-dependent dephosphorylation upon addition of Nebivolol.

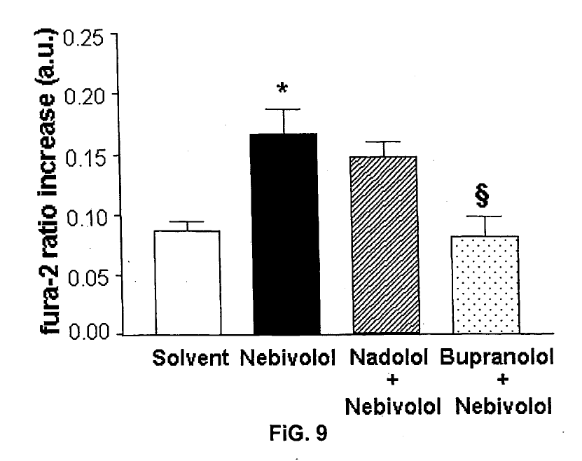

As shown on FIG. 9, a cytosolic calcium increase was evoked by addition of Nebivolol in Baecs that was sensitive to bupranolol (a non-specific beta-blocker), but unchanged by nadolol (a selective beta 1,2-antagonist)

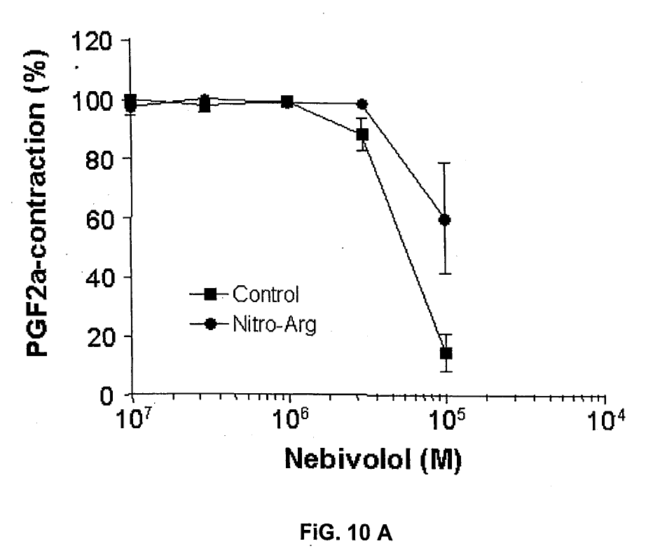

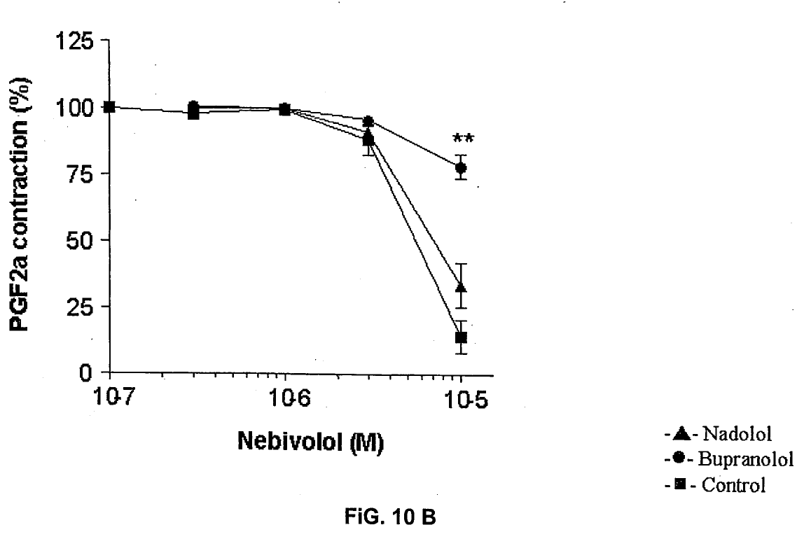

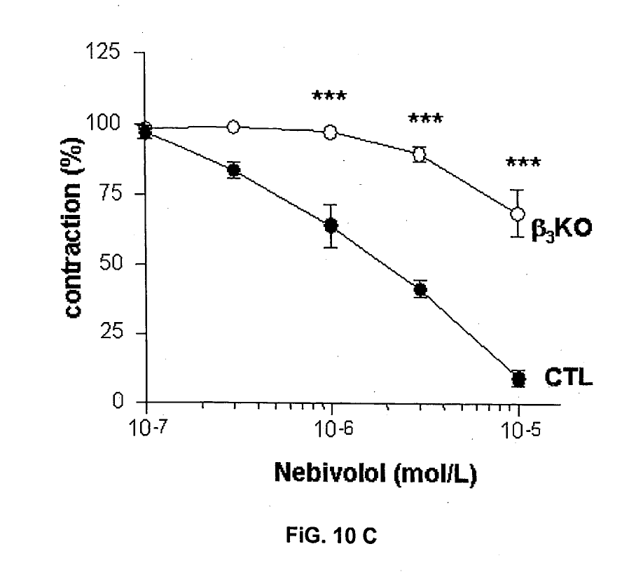

As shown on FIG. 10 nebivolol evoked a dose-dependent-relaxation of rat coronary microvessels that is sensitive to NOS inhibition (A), sensitive to bupranolol but not to nadolol (B). Relaxation to Nebivolol was blunted in coronary microarteries from mice deficient for the beta3-adrenoceptor (C).

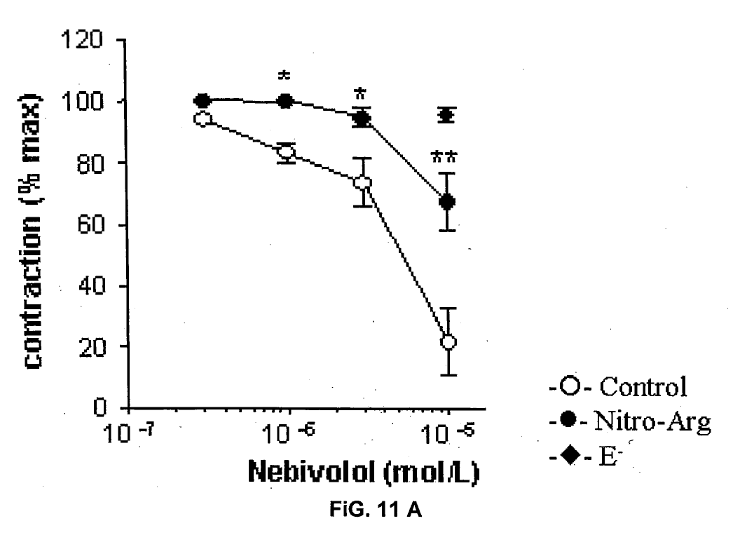

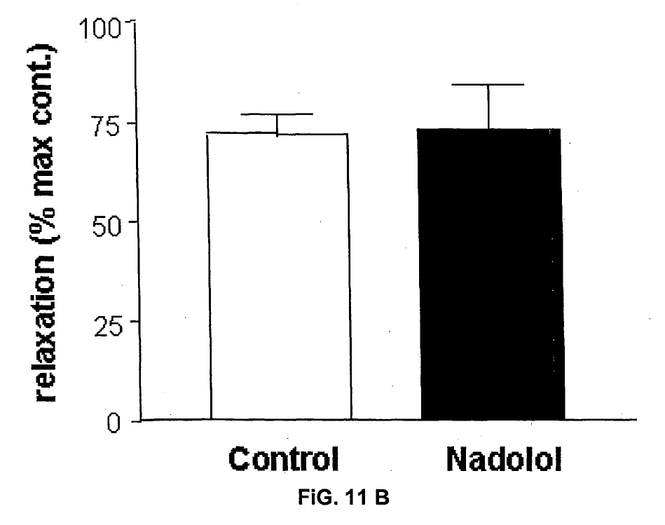

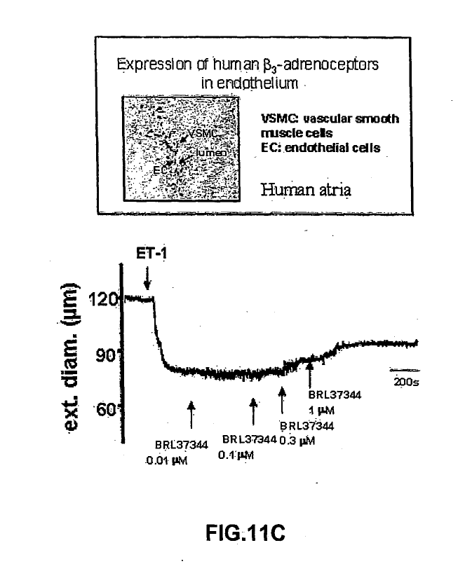

As shown on FIG. 11 nebivolol evoked a dose-dependent-relaxation of human coronary microvessels that is dependent on a functional endothelium, sensitive to NOS inhibition (A), insensitive to nadolol (B) and reproducible by a beta3 preferential agonist (C).

In conclusion, these data demonstrate that nebivolol dilates human coronary resistance arteries through an agonist effect on the endothelial beta3-AR to release NO. This property is particularly beneficial for the treatment of ischemic and failing cardiac diseases.

### Example 3

This example demonstrates pro-angiogenic effects of a beta3-adrenoceptor agonist, as illustrated in FIG. 12. FIG. 12 illustrates that aortic rings from C57BI6 mice treated in vitro with beta3-adrenoceptor agonist SR58611 exhibited a time-dependent increase in new tubes formation compared to untreated controls (CTL). Treatment with the angiogenic cytokine, VEGF, produced a similar effect. In FIG. 12 it is further shown that rings from mice genetically deficient in beta3 adrenoceptor (beta 3-KO mice) do exhibited an angiogenic response to VEGF, but the effect of the beta3-AR agonist, SR58611 was completely lost. These results illustrate the pro-angiogenic effect of a beta3 AR agonist (i.e. SR58611) on the mouse aortic ring.

### Example 4

This example illustrates the infection of human microvascular endothelial cells with an adenovirus encoding the human beta3 AR (Avv) at different MOI (multiplicity of infections). As can be seen on FIG. 13, compared with the negative controls encoding the GFP, the beta3AR Aav dose-dependently induced the expression of specific proteins with immunoaffinity for a specific antibody against the human beta3 AR. The variously sized bands correspond to glycosylated forms of the receptor. The lower panel illustrates the densitometric quantification of the immunoblot signals.

Furthermore, as illustrated on FIGS. 14 and 15 overexpression of human beta3 AR may induce the activation of downstream signalling pathways. From FIG. 14 it can be derived that infection of endothelial cells with Aav encoding the human beta3 AR results in phosphorylation of the protein kinase B (Akt), whereas no activation is seen with a virus encoding the GFP. The lower panel of FIG. 14 illustrates the densitometric quantification of the immunoblotting signals normalized to total Akt proteins.

From FIG. 15 it can be derived that infection of endothelial cells with Aav encoding the human beta3 AR results in phosphorylation of ERK1/2, whereas no activation is seen with a virus encoding the GFP. The lower panel of FIG. 15 illustrates the densitometric quantification of the immunoblotting signals normalized to total ERK proteins.

These results show that overexpression of human beta3 AR in human endothelial cells (with an adenovirus, Aav, FIG. 13) results in activation of downstream activation of specific kinases pathways (Akt, FIG. 14; ERK1/2, FIG. 15) that are known to mediate neo-angiogenesis but also to result in anti-apoptotic effects and generally, protect the endothelium against all major risk factors for cardiovascular diseases.

As illustrated on FIGS. 14 and 15 similar (Akt, eNOS) pathways are activated in cardiac myocytes. This is particularly important since upregulation of eNOS and its activation in cardiac myocytes results in increased biogenesis of mitochondria. These increased mitochondria, in turn, will confer protection of the heart against metabolic deficiency in heart failure and also prevent the occurrence of adverse metabolic remodeling in the metabolic syndrome (characterized by insulin resistance, accumulation of triglycerides and myocardial dysfunction). Therefore, it is submitted that activation and/or overexpression of the beta3 AR in the myocardium confers protection against metabolic remodelling and myocardial dysfunction in states of insulinoresistance, particularly during the metabolic syndrome.

### Example 5

This example (FIG. 16) illustrates that adenoviral expression of the human beta3 AR in cardiac myocytes from C57BI6 control mice or mice overexpressing a cardiac-specific eNOS transgene induces activation of downstream signaling resulting in phosphorylation of Akt and eNOS. Aav infection of neonatal cardiac myocytes resulted in expression of the human beta3AR (different glycosylated proteins, middle panel). Constitutive activation of the receptor or agonist-mediated activation (Isoproterenol+nadolol) resulted in increased phosphorylation of Akt and downstream eNOS in cardiac myocytes from the two strains. It is noted that in wild-type myocytes, overexpression/activation of beta3 AR resulted in increased eNOS expression.

These and previous data demonstrate that the production of NO in the microvessels of the heart has a favorable effect on ventricular relaxation which results in improved heart function. In addition, it was shown that in isolated cardiac myocytes expressing a heterologous human beta3 AR showing activation of downstream signaling results in the phosphorylation of eNOS (endothelial type nitric oxide synthase). These results directly link the function of beta3 AR, activated by agonists, such as isoproterenol, to NO production in the cardiac myocytes (known to exert protective biological effects in the heart) (see FIG. 16).

### REFERENCES

1. Monopoli A, Conti A, Forlani A et al. Beta1 and beta2 adrenoceptors are involved in mediating vasodilation in the human coronary artery. Pharmacol Res. 1993; 27:273-279.
2. Bea M L, Ghaleh B, Giudicelli J F et al. Lack of importance of NO in beta-adrenoceptor-mediated relaxation of large epicardial canine coronary arteries. Br J. Pharmacol. 1994; 111:981-982.
3. Sun D, Huang A, Mital S et al. Norepinephrine elicits beta2-receptor-mediated dilation of isolated human coronary arterioles. Circulation. 2002; 106:550-555.
4. Chang H Y. Role of nitric oxide in vasodilator response induced by salbutamol in rat diaphragmatic microcirculation. Am J. Physiol. 1997; 272:H2173-H2179.
5. Gray D W, Marshall I. Novel signal transduction pathway mediating endothelium-dependent beta-adrenoceptor vasorelaxation in rat thoracic aorta. Br J. Pharmacol. 1992; 107:684-690.
6. Majmudar N G, Anumba D, Robson S C et al. Contribution of nitric oxide to beta2-adrenoceptor mediated vasodilatation in human forearm arterial vasculature. Br J Clin Pharmacol. 1999; 47: 173-177.
7. Strosberg A D. Structural and functional diversity of beta-adrenergic receptors. Ann N Y Acad Sci. 1995; 757:253-260.
8. Emorine L J, Marullo S, Briend-Sutren M M et al. Molecular characterization of the human beta3-adrenergic receptor. Science. 1989; 245:1118-1121.
9. Galitzky J, Reverte M, Portillo M et al. Coexistence of beta1-, beta2-, and beta3-adrenoceptors in dog fat cells and their differential activation by catecholamines. Am J. Physiol. 1993; 264:E403-E412.
10. Gauthier C, Leblais V, Kobzik L et al. The negative inotropic effect of beta3-adrenoceptor stimulation is mediated by activation of a nitric oxide synthase pathway in human ventricle. J Clin Invest. 1998; 102:1377-1384.
11. Moniotte S, Kobzik L, Feron O et al. Upregulation of beta(3)-adrenoceptors and altered contractile response to inotropic amines in human failing myocardium. Circulation. 2001; 103:1649-1655.
12. Donckier J E, Massart P E, Van Mechelen H et al. Cardiovascular effects of beta3-adrenoceptor stimulation in perinephritic hypertension. Eur J Clin Invest. 2001; 31:681-689.
13. Shen Y T, Zhang H, Vatner S F. Peripheral vascular effects of beta-3 adrenergic receptor stimulation in conscious dogs. J Pharmacol Exp Ther. 1994; 268:466-473.
14. Berlan M, Galitzky J, Bousquet-Melou A et al. Beta-3 adrenoceptor-mediated increase in cutaneous blood flow in the dog. J Pharmacol Exp Ther. 1994; 268:1444-1451.
15. Trochu J N, Leblais V, Rautureau Y et al. Beta3-adrenoceptor stimulation induces vasorelaxation mediated essentially by endothelium-derived nitric oxide in rat thoracic aorta. Br J. Pharmacol. 1999; 128:69-76.
16. Rautureau Y, Toumaniantz G, Serpillon S et al. Beta3-adrenoceptor in rat aorta: molecular and biochemical characterization and signalling pathway. Br J. Pharmacol. 2002; 137:153-161.
17. Shimokawa H, Yasutake H, Fujii K et al. The importance of the hyperpolarizing mechanism increases as the vessel size decreases in endothelium-dependent relaxations in rat mesenteric circulation. J Cardiovasc Pharmacol. 1996; 28:703-711.
18. Urakami-Harasawa L, Shimokawa H, Nakashima M et al. Importance of endothelium-derived hyperpolarizing factor in human arteries. J Clin Invest. 1997; 100:2793-2799.
19. Nishikawa Y, Stepp D W, Chilian W M. Nitric oxide exerts feedback inhibition on EDHF-induced coronary arteriolar dilation in vivo. Am J Physiol Heart Circ Physiol. 2000; 279: H459-H465.
20. Thollon C, Fournet-Bourguignon M P, Saboureau D et al. Consequences of reduced production of NO on vascular reactivity of porcine coronary arteries after angioplasty: importance of EDHF. Br J. Pharmacol. 2002; 136:1153-1161.
21. Miura H, Gutterman D D. Human coronary arteriolar dilation to arachidonic acid depends on cytochrome P-450 monooxygenase and Ca²⁺-activated K+ channels. Circ Res. 1998; 83:501-507.
22. Terata K, Miura H, Liu Y et al. Human coronary arteriolar dilation to adrenomedullin: role of nitric oxide and K(+) channels. Am J Physiol Heart Circ Physiol. 2000; 279:H2620-H2626.
23. Moniotte S, Vaerman J L, Kockx M M et al. Real-time RT-PCR for the detection of beta-adrenoceptor messenger RNAs in small human endomyocardial biopsies. J Mol Cell Cardiol. 2001; 33:2121-2133.
24. Sonveaux P, Brouet A, Havaux X et al. Irradiation-induced angiogenesis through the up-regulation of the nitric oxide pathway: implications for tumor radiotherapy. Cancer Res. 2003; 63: 1012-1019.
25. Busse R, Edwards G, Feletou M et al. EDHF: bringing the concepts together. Trends Pharmacol Sci. 2002; 23:374-380.
26. Sofola O A, Knill A, Hainsworth R et al. Change in endothelial function in mesenteric arteries of Sprague-Dawley rats fed a high salt diet. J. Physiol. 2002; 543:255-260.
27. Miura H, Liu Y, Gutterman D D. Human coronary arteriolar dilation to bradykinin depends on membrane hyperpolarization: contribution of nitric oxide and Ca2+-activated K+ channels. Circulation. 1999; 99:3132-3138.
28. Liggett S B, Freedman N J, Schwinn D A et al. Structural basis for receptor subtype-specific regulation revealed by a chimeric beta 3/beta 2-adrenergic receptor. Proc Natl Acad Sci USA. 1993; 90:3665-3669.

1. A method for treating and/or preventing cardiovascular diseases and diseases related thereto, comprising administering a therapeutically effective amount of one or more first compound(s) having a beta3-adrenoceptor agonistic effect and a therapeutically effective amount of one or more second compound(s) having a beta1/beta2-adrenoceptor antagonistic effect to an individual in need thereof wherein said one or more first compound(s) and said one or more second compound(s) are used as combined preparation for simultaneous, separate or sequential use.

2. The method according to claim 1, wherein said treatment and/or prevention is further defined as treating and/or preventing arterial diseases and/or diseases related thereto.

3. The method according to claim 2, wherein said arterial diseases comprise coronary, peripheral or cerebral artery diseases.

4. The method according to claim 1, wherein said treatment and/or prevention is further defined as treating and/or preventing ischemic and failing cardiac diseases and/or diseases related thereto.

5. The method according to claim 4, wherein said failing cardiac disease comprises heart failure.

6. The method according to claim 5, for protecting the heart against metabolic deficiency in heart failure.

7. The method according to claim 1, wherein said treatment and/or prevention is further defined as treating and/or preventing one or more conditions related to metabolic syndrome.

8. The method according to claim 7, wherein said conditions comprise metabolic remodeling and/or myocardial dysfunction.

9. A method for treating and/or preventing failing cardiac diseases and/or diseases related thereto comprising administering a therapeutically effective amount of a compound having a beta3-adrenoceptor agonistic effect to an individual in need thereof.

10. The method according to claim 9, wherein said failing cardiac disease comprises heart failure, and preferably diastolic heart failure.

11. The method according to claim 10, for the preparation of a medicament for protecting the heart against metabolic deficiency in heart failure.

12. The method according to claim 9, wherein said treatment and/or prevention is further defined as treating and/or preventing one or more conditions related to metabolic syndrome.

13. The method according to claim 12, wherein said conditions comprise metabolic remodeling and/or myocardial dysfunction.

14. A method for stimulating neo-angiogenesis and/or for treating and/or preventing angiogenesis-related diseases comprising administering a therapeutically effective amount of a compound having a beta3-adrenoceptor agonistic effect to an individual in need thereof.

15. The method according to claim 14, wherein said compound has a beta3-adrenoceptor agonistic effect and a beta1/beta2-adrenoceptor antagonistic effect.

16. The method according to claim 14, wherein a combination of one or more first compound(s) having a beta3-adrenoceptor agonistic effect with one or more second compound(s) having a beta1/beta2-adrenoceptor antagonistic effect is used as a combined preparation for simultaneous, separate or sequential use.

17. The method according to any one of claims 1, 9, and 14, wherein a compound having a beta3-adrenoceptor agonistic effect is a specific or a non-specific beta3-adrenoceptor agonist.

18. The method according to claim 17, wherein said compound having a beta3-adrenoceptor agonistic effect is selected from the group consisting of norepinephrine, epinephrine, BRL37344, SR 58611, CGP12177, nebivolol, isoproterenol, oxprenolol, carazolol, prenalterol, salbutamol, salmeterol, fenoterol, clenbuterol, +/−trimetoquinol, BRL28410, LY79771, LY362884, LY377604, CL 316243, CP331679, CP331684, AD9677, BMS196085, BMS187257, pindolol, (S)-(−)pindolol, ZD 7114, L755507, L749372, L750355, L757793, L760087, L764646, L766892, L770644, L771047, SMI 1044, SB251023, SB226552, SB229432, SB236923, SB246982, ICI201651, or a pharmacologically acceptable derivative thereof and any combinations thereof.

19. A pharmacological composition comprising a therapeutically effective amount of a first compound having a beta3-adrenoceptor agonistic effect and a therapeutically effective amount of a second compound having a beta1/beta2-adrenoceptor antagonistic effect as a combined preparation for simultaneous, separate or sequential use for treating and/or preventing cardiovascular diseases and diseases related thereto selected form from the group consisting of arterial diseases, ischemic and failing cardiac diseases, one or more conditions related to metabolic syndrome, and any diseases related thereto.

20. A pharmacological composition comprising a therapeutically effective amount of a first compound having a beta3-adrenoceptor agonistic effect and a therapeutically effective amount of a second compound having a beta1/beta2-adrenoceptor antagonistic effect as a combined preparation for simultaneous, separate or sequential use for stimulating neo-angiogenesis and/or for treating and/or preventing angiogenesis-related diseases.

21. The method of claim 5, wherein said heart failure comprises diastolic heart failure.

22. The method according to claim 10, wherein the heart failure comprises diastolic heart failure
